(19) United States
(12) Patent Application Publication
(10) Pub. No.: US 2019/0125808 A1
(43) Pub. Date: May 2, 2019

(54) METHODS OF PREVENTING, TREATING AND DETECTING COLORECTAL CANCER USING BUTYRATE PRODUCING BACTERIA

(71) Applicant: THE TRUSTEES OF COLUMBIA UNIVERSITY IN THE CITY OF NEW YORK, New York, NY (US)

(72) Inventors: W. Ian Lipkin, New York, NY (US); Brent L. Williams, New York, NY (US); Mara Couto-Rodriguez, New York, NY (US)

(21) Appl. No.: 16/089,209

(22) PCT Filed: Mar. 29, 2017
(86) PCT No.: PCT/US17/24718

§ 371 Date: Sep. 27, 2018

Related U.S. Application Data
(60) Provisional application No. 62/315,371, filed on Mar. 30, 2016.

(51) Int. Cl. A61K 35/741; A61K 31/733; C12Q 1/689; A61K 9/00

(52) U.S. Cl. CPC A61K 35/741; A61K 9/0031; C12Q 1/689; A61K 31/733

(57) ABSTRACT

The invention provides for methods for treating and preventing colorectal cancer, non-dysplastic serrated polyps, or serrated crypt foci in a subject by administering butyrate producing bacteria, dietary fiber, sodium butyrate, or a combination thereof. The invention also provides methods for detecting, determining and/or diagnosing colorectal cancer, non-dysplastic serrated polyps, or serrated crypt foci in a subject by detection of the level of butyrate producing bacteria in a sample, as well as the alpha diversity, the operational taxonomic units, the level of bacteria Lactobacillales, and the level of CoA and BcoA nucleic acid.

### CROSS-REFERENCE TO RELATED APPLICATIONS

The present application claims priority of U.S. Provisional Application Ser. No. 62/315,371, filed Mar. 30, 2016, which is hereby incorporated by reference in its entirety.

### FIELD OF THE INVENTION

This invention relates to methods of detection, diagnosis, risk assessment, prevention and treatment of intestinal serrated tumors, colorectal cancer and CIMP colorectal cancer.

### BACKGROUND OF THE INVENTION

Colorectal cancer (CRC) is the second leading cause of cancer-related deaths in the United States and the third most common cancer in men and in women (Siegel, et al. 2013). The majority of CRCs develop along at least two different morphological progression routes: 1) the traditional adenoma-carcinoma sequence pathway; or 2) the more recently recognized serrated neoplasia pathway. The serrated neoplasia pathway comprises a morphologically distinct group of neoplasms and accounts for approximately 35% of CRCs (Jass 2007; Snover 2011). Despite this high incidence, the molecular evolution of serrated lesions is poorly understood. Early lesions in this pathway are non-dysplastic serrated polyps or serrated aberrant crypt foci. These early lesions may progress to sessile serrated adenomas/polyps, serrated adenomas and to serrated carcinoma with unique histological and genetic and epigenetic characteristics (Bettington, et al. 2013). The progression from non-dysplastic serrated polyps to advanced neoplasms is associated with accumulating epigenetic changes. In a subgroup of CRCs, the CpG island methylator phenotype (CIMP), widespread DNA hypermethylation occurs at CpG islands in the promoters of tumor suppressor and DNA repair genes. CIMP accounts for approximately 8-20% of all CRC cases. Higher levels of CpG island methylation are associated with advancing histological stage (O'Brien, et al. 2006; Sawyer, et al. 2002; Vaughn, et al. 2010). CIMP tumors have been associated with an increased risk of CRC-related deaths compared to tumors with chromosomal instability and also may be more likely to be missed by endoscopy or progress rapidly between colonoscopy intervals (Simons, et al. 2013; Nishihara, et al. 2013). Other features associated with CIMP include older age at diagnosis, proximal location in the colon, poor differentiation, and mucinous histology (Toyota, et al. 1999; Hawkins, et al. 2002; van Rijnsoever, et al. 2002; Lee, et al. 2008). The underlying cause of DNA hypermethylation associated with progression of CIMP tumors is unknown.

Activating mutations of BRAF and KRAS are thought to be the earliest genetic alterations in serrated lesions, thus, activation of the RAS-RAF-MEK-ERK-MAP (R-R-M-E-M) pathway may be an instigating signal in serrated carcinogenesis (Rosenburg, et al. 2007; Yang, et al. 2004; Beach, et al. 2005; Chan, et al. 2003; Velho, et al. 2008; Minoo, et al. 2006; Arrington, et al. 2012; Ogino, et al. 2006; Weisenberger, et al. 2006). R-R-M-E-M is an important cellular network that plays a prominent role in the control of cell differentiation, proliferation, survival and apoptosis. Although activation of this pathway alone appears to be sufficient to initiate premalignant serrated hyperplasia, it is not sufficient to induce malignant transformation because activating mutations in this pathway lead to oncogene-induced senescence (OIS) (Bennecke, et al. 2012; Bongers, et al. 2012 Carragher, et al. 2010; Rad, et al. 2013). OIS in the serrated route to CRC appears to be dependent on upregulation of the tumor suppressor, p16^{Ink4a} which arrests cells in G1 phase of the cell cycle. The importance of p16^{Ink4a} in the serrated pathway is underscored by studies that have demonstrated increased expression of p16^{Ink4a} in premalignant serrated lesions and loss of p16^{Ink4a} during malignant transformation (Kriegl, et al. 2011). Furthermore, mouse models of R-R-M-E-M activation through activation of epidermal growth factor receptor (EGFR) signaling and gain of function BRAF and KRAS mutations all lead to serrated hyperplasia, but do not progress to invasive serrated carcinomas in the colon because of p16^{Ink4a}-induced OIS (Bennecke, et al. 2012; Bongers, et al. 2012 Carragher, et al. 2010; Rad, et al. 2013). In human serrated lesions, it appears that senescence is bypassed when p16^{Ink4a} expression is lost as a result of aberrant CpG island methylation of the CDKN2A promoter, contributing to malignant transformation (Kriegl, et al. 2011). The genetic, physiological, or environmental factors that cause some serrated polyps to overcome senescence in this methylation-dependent fashion and develop into malignant cancers are unknown. Without being bound by theory, it is possible that the unidentified factors that lead to CIMP are derived from bacteria.

Approximately 100 trillion bacteria, represented by 500-1000 species and encoding 100 times the number of genes found in the human genome, make up the intestinal microbiome (Qin, et al. 2010). These bacteria perform diverse physiological functions in humans, including regulating carbohydrate fermentation and absorption, maintaining pathogen resistance, stimulating immune system development, and detoxifying carcinogens (Williams, et al. 2011; Hope, et al. 2005). Increasing evidence indicates that bacteria can also have epigenetic effects through chromatin-associated complexes, noncoding RNAs, RNA splicing factors, and histone modifications and DNA methylation (Bierne, et al. 2012). These effects may benefit or harm the host. For example, commensal bacteria have been shown to contribute to intestinal symbiosis by preventing excessive inflammatory reactions through promoter methylation of the Toll-like receptor 4 gene (Takahashi, et al. 2011). Bacterial infection or disruption of commensal populations may induce aberrant DNA methylation, potentially contributing to disease. An example of the latter is Helicobacter pylori, which induces aberrant DNA methylation in the human gastric mucosa at promoters of genes found methylated in gastric cancer cells. These genes include tumor-suppressor genes, DNA repair genes, as well as CpG islands of miRNA genes (Nakajima, et al. 2009; Yoshida, et al. 2013; Sepulveda, et al. 2010). One of the bacterial products with a well-established capacity for regulating epigenetic modifications of DNA is butyrate.

Butyrate is a short chain fatty acid (SCFA) that is produced as a byproduct of dietary fiber fermentation by a subgroup of intestinal bacteria. Butyrate can reach millimolar concentrations in the colonic lumen. In the human colorectum, butyrate is produced through two bacterial pathways: the butyryl-CoA:acetate CoA-transferase (BcoA) pathway and the butyrate kinase pathway. The BcoA pathway accounts for the majority of intestinal butyrate production, thus, levels of the BcoA gene serve as a surrogate estimator of the butyrate-producing capacity of the microbiota (Louis, et al. 2004; Louis and Flint 2007). The most abundant butyrate-producing bacteria (BPB) in the colon belong to clostridial clusters XIVa (i.e. Roseburia spp. and Eubacterium spp.) and IV (i.e. Faecalibacterium prausnitzii). Clostridial clusters XIVa and IV, many of which are BPB, are highly responsive to diet and frequently diminish with age (Duncan, et al. 2007; Hippe, et al. 2011; Neyrinck, et al. 2012; Walker, et al. 2011; Claesson, et al. 2011; Biagi, et al 2010; Claesson, et al. 2012). Diet and aging are two factors strongly linked to CRC risk (Stegeman, et al. 2013). Intriguingly, high dietary fiber is associated with reduced risk of developing a CIMP tumor (Slattery, et al. 2007). However, the mechanism linking dietary fiber to CIMP methylation has not been identified.

Butyrate plays a multifunctional role in maintaining intestinal homeostasis. It can exert various anti-neoplastic effects, including inducing apoptosis and differentiation, inhibiting proliferation, the cell cycle, and angiogenesis, and upregulating detoxifying enzymes (Leonel and Alvarez-Leite 2012; Topping and Clifton 2001; Canani, et al. 2011). One function of butyrate associated with its anti-neoplastic effects is its ability to inhibit the activity of histone deacetylases (HDACs). Butyrate inhibits nearly all class I and class II HDACs, which catalyze the removal of acetyl groups from core histones that are essential to DNA packaging into the basic unit of chromatin, the nucleosome. The transcriptional status of DNA is mediated through protein complexes that interact with and epigenetically alter the nucleosome to either promote or inhibit transcription (Canani, et al. 2012; Yasui, et al. 2003). Epigenetic modification of chromatin by histone deacetylation and CpG island methylation are intimately linked events involved in gene silencing. HDACs associate with nearly all of the methylation machinery, including DNMT1, DNMT3a, DNMT3b and the methyl-binding proteins (MBPs) (MeCP2, MBD1, MBD2, and MBD3) (Rountree, et al. 2001; Bachman, et al. 2001). HDACs form part of multiprotein repressor complexes such as Sin3, CoREST, and NuRD. Recently, NuRD has been shown to cooperate with DNMT1 and DNMT3b in maintaining silencing of a set of tumor suppressor genes (TSGs) in CRC cells (Cai, et al. 2013). HDAC inhibitors (HDACi), including butyrate, may also affect DNA methylation through the regulation of DNMTs. HDACi have been shown to down-regulate both DNMT1 and DNMT3B (Sarkar, et al. 2011). Previous studies have shown that butyrate inhibits ERK phosphorylation, which leads to decreased DNMT1 expression (Sarkar, et al. 2011; Wang, et al. 2010; Ding, et al. 2001). This may provide additional evidence for the importance of BPB in serrated tumors since EGFR activation and activating mutations in BRAF and KRAS mediate their cellular proliferative effects through downstream phosphorylation of ERK (Bennecke, et al 2012; Bongers, et al. 2012 Carragher, et al. 2010; Rad, et al. 2013). Furthermore, HDACi, including butyrate, down-regulate the expression of EGFR (upregulation of EGFR gene occurs in 60-80% of CRCs) (Chou, et al. 2011; Cunningham, et al. 2004; Daly and Shirazi-Beechey 2006).

The present invention describes the contribution of the intestinal microbiota to CIMP and microbial signatures that are distinct in CIMP patients as compared to Non-CIMP patients. Specifically, significant loss of bacterial diversity in intestinal tissues from individuals harboring CIMP tumors was associated with significant deficiencies in butyrate-producing bacterial (BPB) populations. This novel finding is the basis for methods of preventing, treating, detecting and diagnosing colorectal cancer.

### SUMMARY OF THE INVENTION

The present invention relates to methods of treatment, prevention, detection, diagnosis, and risk assessment of intestinal serrated tumors, colorectal cancer and CpG island methylator phenotype (CIMP) colorectal cancer based upon the surprising discovery that butyrate producing bacteria (BPB) was significantly reduced in patients with CIMP tumors.

In certain aspects, the invention provides a method of treating and/or preventing colorectal n cancer, non-dysplastic serrated polyps, or serrated crypt foci in a subject comprising administering butyrate producing bacteria, dietary fiber, or a combination thereof, to a subject. In some embodiments, the dietary fiber is inulin fiber. In some embodiments, butyrate producing bacteria and inulin fiber are administered to the subject. In some embodiments, the methods further comprise administering sodium butyrate to the subject. In some embodiments, the colorectal cancer is CpG island methylator phenotype (CIMP). In some embodiments, the colorectal cancer is a serrated neoplasia.

In some embodiments, the level of butyrate producing bacteria is measured in a sample from the subject before administration. In some embodiments, the butyrate producing bacteria, dietary fiber, sodium butyrate, or a combination thereof, is administered to the subject if the level of butyrate producing bacteria in the sample from the subject is reduced compared to the level of butyrate producing bacteria in a sample from a healthy subject.

In some embodiments, the alpha diversity of the bacteria is measured in a sample from the subject before administration. In some embodiments, the butyrate producing bacteria, dietary fiber, sodium butyrate, or a combination thereof, is administered to the subject if the alpha diversity of the sample from the subject is reduced compared to the alpha diversity of a sample from a healthy subject.

In some embodiments, the operational taxonomic units of the bacteria are measured in a sample from the subject before administration. In some embodiments, the butyrate producing bacteria, the dietary fiber, or a combination thereof, is administered to the subject if the operational taxonomic units (OTUs) of the sample from the subject are reduced compared to the OTUs of a sample from a healthy subject.

In some embodiments the level of bacteria belonging to the order Lactobacillales in a sample from the subject is measured before administration. In some embodiments, the butyrate producing bacteria, dietary fiber, sodium butyrate, or a combination thereof, is administered to the subject if the level of bacteria belonging to the order Lactobacillales in the sample from the subject is increased compared to the level of bacteria belonging to the order Lactobacillales in a sample from a healthy subject.

In some embodiments, the level of bacterial butyryl coenzyme A genes (CoA), bacterial acetate CoA transferase genes (BcoA), or a combination thereof, is measured in a sample from the subject before administration. In some embodiments, the butyrate producing bacteria, dietary fiber, sodium butyrate, or a combination thereof, is administered to the subject if the level of BcoA of the sample from the subject is reduced compared to the level of BcoA of the sample from a healthy subject. In some embodiments, the BcoA is the BcoA gene from Faecalibacterium prausnitzii. In some embodiments, the BcoA is the BcoA gene from the genus Roseburia. In some embodiments, the BcoA is the BcoA gene from the genus Eubacterium. In some embodiments, the BcoA is the BcoA gene from Clostridium sp. SS2/1. In some embodiments, the butyrate producing bacteria, dietary fiber, or a combination thereof, is administered to the subject if the level of CoA of the sample from the subject is reduced compared to the level of CoA of the sample from a healthy subject.

In some embodiments, the cecal levels of short chain fatty acids are measured in a sample from the subject before administration. In some embodiments, the short chain fatty acid is butyrate. In some embodiments, the butyrate producing bacteria, dietary fiber, sodium butyrate, or a combination thereof, is administered to the subject if the level of cecal levels of short chain fatty acids in the sample from the subject are reduced compared to the level of cecal levels of short chain fatty acids in a sample from a healthy subject.

In certain aspects, the invention provides a method of treating a subject with a deficiency in butyrate producing bacteria comprising administering butyrate producing bacteria, dietary fiber, or a combination thereof to a subject. In some embodiments, the dietary fiber is inulin fiber. In some embodiments, butyrate producing bacteria and inulin fiber are administered to the subject. In some embodiments, the methods of treating a subject with a deficiency in butyrate producing bacteria further comprise administering sodium butyrate to the subject.

In other aspects, the present invention also provides for methods of detecting and diagnosing colorectal cancer, non-dysplastic serrated polyps, or serrated crypt foci in a subject via the detection and/or measurement of certain bacterium as well as other parameters. Specifically, the invention provides for the following methods.

In certain aspects, the invention provides a method of detecting or diagnosing colorectal cancer, non-dysplastic serrated polyps, or serrated crypt foci in a subject comprising measuring the level of butyrate producing bacteria in a sample from the subject, wherein colorectal cancer, non-dysplastic serrated polyps, or serrated crypt foci is detected or diagnosed, if the level of butyrate producing bacteria in the sample from the subject is reduced or altered compared to the level of butyrate producing bacteria in a sample from a healthy subject.

In certain aspects, the invention provides a method of detecting or diagnosing colorectal cancer, non-dysplastic serrated polyps, or serrated crypt foci in a subject comprising measuring the alpha diversity of bacteria in a sample from the subject, wherein colorectal cancer, non-dysplastic serrated polyps, or serrated crypt foci is detected or diagnosed, if the alpha diversity in the sample from the subject is reduced or altered compared to the alpha diversity in a sample from a healthy subject.

In certain aspects, the invention provides a method of detecting or diagnosing colorectal cancer, non-dysplastic serrated polyps, or serrated crypt foci in a subject comprising measuring the operational taxonomic units (OTUs) of the bacteria in a sample from the subject, wherein colorectal cancer, non-dysplastic serrated polyps, or serrated crypt foci is detected or diagnosed, if the OTUs in the sample from the subject is reduced or altered compared to the OTUs in a sample from a healthy subject.

In certain aspects, the invention provides a method of detecting or diagnosing colorectal cancer, non-dysplastic serrated polyps, or serrated crypt foci in a subject comprising measuring the level of bacteria belonging to the order Lactobacillales in a sample from the subject, wherein colorectal cancer, non-dysplastic serrated polyps, or serrated crypt foci is detected or diagnosed, if the level of bacteria belonging to the order Lactobacillales in the sample from the subject is increased or altered compared to the level of bacteria belonging to the order Lactobacillales in a sample from a healthy subject.

In certain aspects, the invention provides a method of detecting or diagnosing colorectal cancer, non-dysplastic serrated polyps, or serrated crypt foci in a subject comprising measuring the level of bacterial butyryl coenzyme A nucleic acid (CoA), bacterial acetate CoA transferase nucleic acid (BcoA), or a combination thereof, in a sample from the subject, wherein colorectal cancer, non-dysplastic serrated polyps, or serrated crypt foci is detected or diagnosed, if the level of BcoA of the sample from the subject is reduced or altered compared to the level of BcoA of the sample from a healthy subject, or if the level of CoA of the sample from the subject is reduced compared to the level of CoA of the sample from a healthy subject.

In certain aspects, the invention provides a method of detecting or diagnosing colorectal cancer, non-dysplastic serrated polyps, or serrated crypt foci in a subject comprising: (a) contacting nucleic acid from a biological sample with at least one primer that specifically hybridizes to the 16S rRNA nucleotide sequence, the BcoA nucleotide sequence, or the CoA nucleotide sequence of a butyrate producing bacteria; (b) subjecting the nucleic acid and the primer to amplification conditions; and (c) measuring the level of amplification product, wherein colorectal cancer, non-dysplastic serrated polyps, or serrated crypt foci is detected or diagnosed, if the level of amplification product in the sample from the subject is reduced or altered compared to the level of amplification product in a sample from a healthy subject.

All of the foregoing methods can further comprise administering butyrate producing bacteria, dietary fiber, or a combination thereof to the subject if the subject is diagnosed with colorectal cancer, non-dysplastic serrated polyps, or serrated crypt foci. In some embodiments, the methods further comprise administering sodium butyrate to the subject.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

Certain abbreviations will be used in the figures including; CIMP—CpG island methylator phenotype; BPB—butyrate producing bacteria; and BcoA—butyryl-CoA:acetate CoA-transferase.

FIGS. 1A and 1B are graphs showing alpha diversity in CIMP and Non-CIMP tumor and normal tissues. FIG. 1A shows all bacteria and FIG. 1B shows Firmicutes phylum, in CIMP and Non-CIMP tissues.

FIG. 2 shows jackknifed UPGMA tree calculated from the unweighted UniFrac distances among samples of CIMP and Non-CIMP tumor and normal tissues (based on the subsampled dataset of 15, 658 16S rDNA sequences/sample).

FIG. 3 shows heatmap of enriched OTUs in CIMP versus Non-CIMP tissues.

FIG. 4A shows a bar chart comparing levels of BPB (in terms of % of total 16S rRNA reads) in CIMP versus Non-CIMP tumor and normal tissues. FIG. 4B shows a bar chart comparing levels of Prevotella spp. (in terms of % of total 16S rRNA reads) in CIMP versus Non-CIMP tumor and normal tissues. Mann-Whitney p-value shown.

FIG. 5 are graphs showing the tumor and normal tissue concentrations of short chain fatty acids, butyrate (FIG. 5A), acetate (FIG. 5B), and propionate (FIG. 5C) in CIMP and non-CIMP tumor and normal tissues. Mann-Whitney p-value shown.

FIGS. 6A and 6B are graphs showing alpha diversity in HBUS and wild type mice of A) all bacteria and B) Firmicutes phylum. FIG. 6C is a heatmap showing abundance of Firmicutes species in wild type and HBUS mice.

FIGS. 7A, 7B and 7C are graphs showing BPB and BcoA quantities in feces of mice on a high-fiber diet and a no-fiber diet. FIG. 7A shows the number of Roseburia/Eubacterimum 16S gene copies, FIG. 7B shows the number of Faecalibacterium prausnitzii 16S gene copies, and FIG. 7C the number of BcoA gene copies (*, p<0.05; **, p<0.01; ***, p<0.001; n.s., not significant; Mann-Whitney U test).

FIG. 8 shows a dietary scheme to evaluate the role of butyrate in CIMP in HBUS mice and expected results.

### DETAILED DESCRIPTION OF THE INVENTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

In accordance with the present invention, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual, 3rd ed. Cold Spring Harbor Laboratory Press: Cold Spring Harbor, N.Y.; Ausubel et al. eds. (2005) Current Protocols in Molecular Biology, John Wiley and Sons, Inc.: Hoboken, N.J.; Bonifacino et al. eds. (2005) Current Protocols in Cell Biology, John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

The singular forms “a,” “an,” and “the” include plural reference unless the context clearly dictates otherwise.

As used herein, the term “subject” means any organism including, without limitation, a mammal such as a mouse, a rat, a dog, a guinea pig, a ferret, a rabbit and a primate. In the preferred embodiment, the subject is a human being.

The term “patient” as used in this application means a human subject. In some embodiments of the present invention, the “patient” is one that has or has been diagnosed with colon or colorectal cancer (e.g., CIMP colorectal cancer), non-dysplastic serrated polyps, or serrated crypt foci. In some embodiments, the “patient” is one that is at risk for colon or colorectal cancer (e.g., CIMP colorectal cancer), non-dysplastic serrated polyps, or serrated crypt foci.

The terms “colon cancer”, “colorectal cancer” and “CRC” will be used interchangeably and mean cancer that involves the large intestine.

The phrase “therapeutically effective amount” is used herein to mean an amount sufficient to cause an improvement in a clinically significant condition in the subject, or delays or minimizes or mitigates one or more symptoms associated with the disease or disorder, or results in a desired beneficial change of physiology in the subject.

The term “detection”, “detect”, “detecting” and the like as used herein means as used herein means to discover or identify the presence or existence of.

The terms “screen” and “screening” and the like as used herein means to test a subject or patient to determine if they have a particular illness or disease, in this case colorectal cancer (e.g., CIMP colorectal cancer), non-dysplastic serrated polyps, or serrated crypt foci. The term also means to test an agent to determine if it has a particular action or efficacy.

The terms “diagnosis”, “diagnose”, diagnosing” and the like as used herein means to determine what physical disease or illness a subject or patient has, in this case colorectal cancer (e.g., CIMP colorectal cancer), non-dysplastic serrated polyps, or serrated crypt foci.

The terms “identification”, “identify”, “identifying” and the like as used herein means to recognize a disease in a subject or patient, in this case colorectal cancer (e.g., CIMP colorectal cancer), non-dysplastic serrated polyps, or serrated crypt foci. The term also means to recognize an agent as being effective for a particular use.

The terms “healthy control” and “healthy subject” are used interchangeably in this application and are a human subject who is not suffering from colorectal cancer (e.g., CIMP colorectal cancer), non-dysplastic serrated polyps, or serrated crypt foci.

The term “control sample” would be a sample from a healthy subject or healthy control. In some embodiments, a “control sample” can be a sample from a subject or control that is not suffering from CIMP colorectal cancer.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “alpha diversity” is defined as mean species diversity in a sample. In most cases, the term is used herein to denote the mean species diversity of bacteria.

The term “operational taxonomic unit” is defined as a group of species clustered according to their similarity to one another, in this case bacteria,

The term “in need thereof” would be a subject known or suspected of having, or being at risk for, colon cancer, colorectal cancer, non-dysplastic serrated polyps, or serrated crypt foci. Risk factors for colon cancer include but are not limited to a personal history of colon cancer or colorectal cancer or polyps or other cancer, a family history of colon cancer or other cancer, certain inherited syndromes, obesity, older age, diabetes and smoking.

The term “agent” as used herein means a substance that produces or is capable of producing an effect and would include, but is not limited to, chemicals, pharmaceuticals, biologics, small organic molecules, antibodies, nucleic acids, peptides, and proteins.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### The Role of the Microbiome and Butyrate in CIMP Colorectal Cancer

In the past two decades, our mechanistic understanding of colorectal cancer (CRC) has been revolutionized by the identification of distinct molecular pathways associated with tumor progression. A group of spontaneous CRCs that develop through the “serrated neoplasia pathway” has been shown to exhibit high levels of promoter methylation of CpG islands (CpG island methylator phenotype, CIMP). The cause of CpG island hypermethylation in CIMP tumors remains unknown. Described herein are results investigating the potential contribution of the intestinal microbiota to CIMP. These results have revealed microbial signatures that are distinct to matched tumor and normal mucosal tissue from CIMP patients compared to Non-CIMP patients. Remarkably, a significant loss of bacterial diversity in intestinal tissues from individuals harboring CIMP tumors was associated with significant deficiencies in butyrate-producing bacterial (BPB) populations.

Without being bound by theory, it is possible that CIMP is promoted in precancerous polyps as a result of a deficiency in intestinal butyrate, a potent histone deacetylase (HDAC) inhibitor with substantial anti-neoplastic effects and the potential to alter the epigenetic landscape. It is further possible that diet can play a major role in CIMP and that fiber-deficient diets promote aberrant methylation in CIMP tumors by inhibiting the growth of BPB and consequently lowering the levels of butyrate in the large intestine.

Significant group-specific differences were found in the microbiota in patients with CIMP tumors (matched tumor and normal biopsies) versus patients with Non-CIMP tumors (matched tumor and normal biopsies). Both BPB and tumor butyrate concentrations were significantly reduced in CIMP patients compared to Non-CIMP patients (in both normal and tumor tissue). Based on these findings, the link between butyrate and epigenetic regulation through HDAC inhibition and DNMT expression, and evidence that butyrate can influence R-R-M-E-M activation and OIS, a model for the role of BPB in the development of CIMP is proposed. EGFR activation and activating KRAS and BRAF mutations lead to increased proliferation through activation of the R-R-M-E-M pathway. If this occurs in a healthy microbiome (where BPB abound as a result of sufficient dietary fiber), butyrate: (i) reduces expression of EGFR; (ii) inhibits ERK regulated enhancement of DNMTs; (iii) inhibits HDAC activity; and (iv) prevents association of DNMTs, HDACs, and MBPs. In the model, butyrate controls and prevents aberrant methylation found in CIMP tumors resulting in active tumor suppressor gene transcription. Butyrate also promotes OIS through p16^{Ink4a} upregulation (Munro, et al. 2004). Thus, butyrate may prevent progression of precancerous lesions specifically carrying KRAS or BRAF mutations. If, however, precancerous lesions carrying KRAS or BRAF mutations develop in a microbiome deficient in BPB (in, for example, a fiber-deficient diet), there is no brake to neoplasia: (i) EGFR expression is increased; (ii) DNMT expression and HDAC are not inhibited by butyrate; (iii) DNA methylation and silencing machinery are aberrantly activated leading to promoter CpG island methylation and tumor suppressor gene silencing, (iv) OIS is overcome; and (v) precancerous lesions progress to malignant CIMP CRC.

Described herein is the first study to investigate how differences in colonic bacteria may mediate functional epigenetic alterations leading to the development of the CIMP CRCs. The use of biopsies rather than fecal material provides the opportunity to specifically compare the microbial structure of tumor tissue with unaffected (normal) tissue from both CIMP and Non-CIMP patients. In addition to examining bacterial structure based on bacterial 16S rDNA sequencing, algorithms for metagenome prediction were applied in order to gain insights into functional modules that differentiate these tumor phenotypes. Additionally, for the first time, concentrations of butyrate and other fatty acids directly in tumor and normal tissue biopsies have been assessed in order to demonstrate the relationships among BPB, butyrate and tumor phenotypes. Through evaluation of structure and functions of the microbiome, combined with the investigation of the role of butyrate in the development of CIMP, this study forms the basis for future work in CRC and perhaps even other cancers.

Methods of Treating and/or Preventing Colorectal Cancer

The present invention provides methods for treating and/or preventing colorectal cancer, non-dysplastic serrated polyps, or serrated crypt foci comprising administering butyrate producing bacteria, dietary fiber, or a combination thereof. In some embodiments, the method further comprises administering sodium butyrate to the subject.

In some embodiments, the colorectal cancer is CpG island methylator phenotype (CIMP). In some embodiments, the colorectal cancer is a serrated neoplasia. In some embodiments the colorectal cancer is an adenocarcinoma, a carcinoid tumor, a gastroinstestinal stromal tumor, a lymphoma, or a sarcoma.

In some embodiments, the subject is already suspected to have colorectal cancer (e.g. CIMP colon cancer). In other embodiments, the subject is being treated for a colorectal cancer, before being treated according to the methods of the invention. In other embodiments, the subject is not being treated for a colorectal cancer, before being treated according to the methods of the invention.

In some embodiments, the subject is at risk for colorectal cancer. Risk factors for colorectal cancer include but are not limited to a personal history of colorectal cancer or polyps or other cancer, a family history of colorectal cancer or other cancer, certain inherited syndromes, obesity, older age, diabetes and smoking.

In a further embodiment, the invention provides methods for treating and/or preventing a digestive system cancer comprising administering butyrate producing bacteria, dietary fiber, or a combination thereof. In some embodiments, the method further comprises administering sodium butyrate to the subject. The digestive system comprises the gastrointestinal tract including structures from the mouth to the anus, and the accessory organs. For example, this includes, but is not limited to, the mouth, the pharynx, the esophagus, the stomach, the small intestine, including the duodenum, jejunum, and ileum, the large intestine including the cecum, colon, and rectum, and the anus. In further embodiments, the accessory organs of the digestive system include, but are not limited to, the liver, the pancreas, and the gall bladder. In one embodiment, the digestive system cancer includes, but is not limited to, mouth cancer, pharynx cancer, esophageal cancer, stomach cancer, small intestine cancer, large intestine cancer, colon cancer, rectal cancer, anal cancer, liver cancer, pancreatic cancer, and gall bladder cancer.

The present invention also provides methods for decreasing tumor growth in a subject comprising administering butyrate producing bacteria, dietary fiber, or a combination thereof. In some embodiments, the method further comprises administering sodium butyrate to the subject. In one embodiment, the tumor is a tumor of the colon (e.g. CIMP colon or colorectal cancer). In one embodiment, the tumor is a tumor of the digestive system. Tumor growth can be measured in a variety of ways, known to one of skill in the art. For example, tumor growth can be measured by measuring the tumor volume over time. Tumor volume can be measured in a variety of ways, known to one of skill in the art including, but not limited to, positron emission tomography and computed tomography (PET-CT), single-photon emission computed tomography (SPECT-CT), magnetic resonance spectroscopy (MR), X-ray computed tomography (CT), and molecular imaging.

The present invention provides methods for treating and/or preventing non-dysplastic serrated polyps, or serrated crypt foci of the colon comprising administering butyrate producing bacteria, dietary fiber, sodium butyrate, or a combination thereof. A polyp is a growth of tissue from the inner lining of the colon into the lumen of the colon. A polyp can be benign or pre-cancerous. A polyp can precede the development of any neoplasm, benign or malignant.

The present invention provides methods for treating and/or preventing dysplasic polyps of the colon in a subject comprising administering butyrate producing bacteria, dietary fiber, sodium butyrate, or a combination thereof. Dysplasia is a condition where there is a morphologically identifiable local tissue abnormality at a given site. Dysplasia can have characteristics including, but not limited to, increased cell number, nuclear abnormalities, and cellular differentiation abnormalities, compared to normal cells. A dysplasia can precede the development of any neoplasm, benign or malignant.

In certain aspects, the invention provides a method of treating a subject with a deficiency in butyrate producing bacteria comprising administering butyrate producing bacteria, dietary fiber, or a combination thereof to a subject. In some embodiments, the method further comprises administering sodium butyrate to the subject.

In some of the preceding methods of the invention, the dietary fiber is inulin fiber. In some embodiments, butyrate producing bacteria and inulin fiber are administered to the subject. In some embodiments, the butyrate producing bacteria, sodium butyrate, and/or dietary fiber is administered orally. In some embodiments, the butyrate producing bacteria is administered rectally.

In some of the preceding methods of the invention, the butyrate producing bacteria administered to the subject belong to clostridial cluster XIVa, clostridial cluster IV, or a combination thereof. In some embodiments, the butyrate producing bacteria administered to the subject belong to the phylum Firmicutes, the phylum Bacteroidetes, or a combination thereof. In some embodiments, the butyrate producing bacteria administered to the subject belong to the genus Roseburia, the genus Eubacterium, the genus Faecalibacterium, the genus Clostridium, the genus Prevotella, or a combination thereof. In some embodiments, the butyrate producing bacteria administered to the subject belong to the genus Roseburia is Roseburia hominis. In some embodiments, the butyrate producing bacteria administered to the subject belong to the genus Roseburia is Roseburia faecis. In some embodiments, the butyrate producing bacteria administered to the subject belong to the genus Eubacterium is Eubacterium rectale. In some embodiments, the butyrate producing bacteria administered to the subject belong to the genus Eubacterium is Eubacterium hallii. In some embodiments, the butyrate producing bacteria administered to the subject belong to the genus Faecalibacterium is Faecalibacterium prausnitzii. In some embodiments, the butyrate producing bacteria administered to the subject belong to the genus Clostridium is Clostridium sp. str., M62/1. In some embodiments, the butyrate producing bacteria administered to the subject belong to the genus Clostridium is Clostridium sp. SS2/1. In some embodiments, more than one butyrate producing bacteria is administered to the subject.

The present invention also provides a kit for treating colorectal cancer (e.g. CIMP colorectal cancer), non-dysplastic serrated polyps, or serrated crypt foci in a subject. In one embodiment, the kit for treating colorectal cancer, non-dysplastic serrated polyps, or serrated crypt foci comprises butyrate producing bacteria, dietary fiber, sodium butyrate, or a combination thereof, to administer to a subject, a means for administering, and instructions of use.

In some embodiments, the level of butyrate producing bacteria in a sample from the subject is measured before administration of butyrate producing bacteria, dietary fiber, sodium butyrate, or a combination thereof. In some embodiments, the butyrate producing bacteria, dietary fiber, or a combination thereof, is administered to the subject if the level of butyrate producing bacteria in the sample from the subject is reduced compared to the level of butyrate producing bacteria in a control sample.

In some embodiments, the level of butyrate producing bacteria is the level of bacteria belonging to clostridial cluster XIVa or clostridial cluster IV. In some embodiments, the level of butyrate producing bacteria is the level of bacteria belonging to the phylum Firmicutes, the phylum Bacteroidetes, or a combination thereof. In some embodiments, the level of butyrate producing bacteria is the level of bacteria belonging to the genus Roseburia, the genus Eubacterium, the genus Faecalibacterium, the genus Clostridium, the genus Prevotella, or a combination thereof. In some embodiments, the level of butyrate producing bacteria is the level of Roseburia hominis, Roseburia faecis, Eubacterium rectale, Eubacterium hallii, Faecalibacterium prausnitzii, Clostridium sp. str., M62/1, Clostridium sp. SS2/1, or a combination thereof.

In some embodiments, the level of butyrate producing bacteria is the level of mucin-degrading bacteria.

In some embodiments, the butyrate producing bacteria, dietary fiber, sodium butyrate, or a combination thereof, is administered to the subject if the level of bacteria belonging to the order Lactobacillales in the sample from the subject is increased compared to the level of bacteria belonging to the order Lactobacillales in a sample from a control sample.

The level of butyrate producing bacteria in the sample can be measured by any technique known in the art including the ones outlined below, including but not limited to 16S rRNA gene sequencing, bacterial 16S rRNA qPCR, RT-PCR, or qRT-PCR, and whole genome sequencing. In some embodiments, the sample is feces. In some embodiments, the sample is a tissue biopsy. In some embodiments, the level of bacteria is measured by the number of copies of bacterial 16S rRNA gene per gram of feces. In some embodiments, the control sample is from a healthy subject, i.e., a subject not suffering from colorectal cancer. In some embodiments, the control sample is from a subject not suffering from CIMP CRC.

In some embodiments, the alpha diversity of the bacteria in a sample from the subject is measured before administration of butyrate producing bacteria, dietary fiber, sodium butyrate, or a combination thereof. In some embodiments, the butyrate producing bacteria, dietary fiber, or a combination thereof, is administered to the subject if the alpha diversity of the sample from the subject is reduced compared to the alpha diversity of a control sample.

The alpha diversity in the sample can be measured by any technique known in the art including the ones outlined below, including but not limited to is measured by bacterial 16S rRNA gene sequencing, alternatively combined with an algorithm. In some embodiments, the sample is feces. In some embodiments, the sample is a tissue biopsy. In some embodiments, the control sample is from a healthy subject, i.e., a subject not suffering from colorectal cancer. In some embodiments, the control sample is from a subject not suffering from CIMP CRC.

In some embodiments, the operational taxonomic units in a sample from the subject are measured before administration butyrate producing bacteria, dietary fiber, sodium butyrate, or a combination thereof. In some embodiments, the butyrate producing bacteria, dietary fiber, or a combination thereof, is administered to the subject if the operational taxonomic units (OTUs) of the sample from the subject are reduced compared to the OTUs of a control.

The OTUs in the sample can be measured by any technique known in the art including the ones outlined below, including but not limited to bacterial 16S rRNA gene sequencing, alternatively combined with an algorithm. In some embodiments, the sample is feces. In some embodiments, the sample is a tissue biopsy. In some embodiments, the control sample is from a healthy subject, i.e., a subject not suffering from colorectal cancer. In some embodiments, the control sample is from a subject not suffering from CIMP CRC.

In some embodiments, the level of bacterial butyryl coenzyme A genes (CoA), bacterial acetate CoA transferase genes (BcoA), or a combination thereof, is measured in a sample from the subject before administration of the butyrate producing bacteria, dietary fiber, sodium butyrate, or a combination thereof. In some embodiments, the butyrate producing bacteria, dietary fiber, or a combination thereof, is administered to the subject if the level of BcoA of the sample from the subject is reduced compared to the level of BcoA of a control sample. In some embodiments, the BcoA is the BcoA gene from Faecalibacterium prausnitzii. In some embodiments, the BcoA is the BcoA gene from the genus Roseburia. In some embodiments, the BcoA is the BcoA gene from the genus Eubacterium. In some embodiments, the BcoA is the BcoA gene from Clostridium sp. SS2/1. In some embodiments, the butyrate producing bacteria, dietary fiber, sodium butyrate, or a combination thereof, is administered to the subject if the level of CoA of the sample from the subject is reduced compared to the level of CoA of the control.

The level of CoA or BcoA can be measured by any technique known in the art including the ones outlined below, including but not limited to qPCR, RT-PCR, and qRT-PCR. In some embodiments, the sample is feces. In some embodiments, the sample is a tissue biopsy. In some embodiments, the level of BcoA is measured by is the number of copies of the BcoA gene per gram of feces. In some embodiments, the control sample is from a healthy subject, i.e., a subject not suffering from colorectal cancer. In some embodiments, the control sample is from a subject not suffering from CIMP CRC.

In some embodiments, the cecal levels of short chain fatty acids are measured in a sample from the subject before administration of the butyrate producing bacteria, dietary fiber, sodium butyrate, or a combination thereof. In some embodiments, the short chain fatty acid is butyrate. The short chain fatty acids can be measured by any technique known in the art including but not limited to electron ionization-GCMS. In some embodiments, the butyrate producing bacteria, dietary fiber, or a combination thereof, is administered to the subject if the cecal levels of short chain fatty acids in the sample from the subject are reduced compared to the cecal levels of short chain fatty acids in a control sample.

### Methods of Detecting, Determining and Diagnosing Colorectal Cancer,

The present invention provides for methods of detecting, determining and/or diagnosing the presence of or a predisposition to colon or colorectal cancer (e.g. CIMP colorectal cancer), non-dysplastic serrated polyps, or serrated crypt foci in a subject. The methods can comprise detecting in a sample from the subject, a reduction or an alteration in the number of butyrate producing bacteria compared to the number of butyrate producing bacteria in a control sample. In one embodiment, the detecting comprises detecting whether there is a reduction or an alteration in the diversity of colonic bacteria. In one embodiment, the detecting comprises detecting a reduction or an alteration in diversity of colonic bacteria due to a reduction or an alteration in the numbers of butyrate producing bacteria.

The present invention also provide a method of detecting and/or determining the presence of, or predisposition to, colorectal cancer (e.g. CIMP colorectal cancer), non-dysplastic serrated polyps, or serrated crypt foci in a subject. In one embodiment, the presence of, or predisposition to colorectal cancer (e.g. CIMP colorectal cancer), non-dysplastic serrated polyps, or serrated crypt foci in a subject is detected or determined by extracting a sample from a subject and detecting the alteration, presence, absence or reduction of butyrate producing bacteria in the sample, wherein alteration, absence, or reduction of butyrate producing bacteria indicates the presence of, or predisposition to, colorectal cancer (e.g. CIMP colorectal cancer), non-dysplastic serrated polyps, or serrated crypt foci. In one embodiment the sample is feces. In another embodiment, the sample is a colon tissue sample. In one embodiment, a reduction or alteration of butyrate producing bacteria in the sample comprises detecting a lower amount of butyrate producing bacteria in the sample than the amount of butyrate producing bacteria in a control sample. In one embodiment, the control sample is from a subject not suffering from colorectal cancer, non-dysplastic serrated polyps, or serrated crypt foci. In a further embodiment, the control sample is from a subject not suffering from CIMP colorectal cancer. In another embodiment, the control sample is not cancer cells. In one embodiment, the butyrate producing bacteria is detected by 16S rRNA gene high throughput sequencing, 16S rRNA qPCR, RT-PCR, or qRT-PCR, CoA gene qPCR, RT-PCR, or qRT-PCR, BcoA gene qPCR, RT-PCR, or qRT-PCR, or whole genome sequencing. In one embodiment, the method further comprises administering butyrate producing bacteria, dietary fiber, sodium butyrate or a combination thereof to the subject where the level of butyrate producing bacteria is altered or reduced.

In certain aspects, the invention provides a method of detecting, determining or diagnosing colorectal cancer, non-dysplastic serrated polyps, or serrated crypt foci in a subject comprising: (a) obtaining a sample from a subject; (b) measuring the level of butyrate producing bacteria in the sample from the subject; (c) comparing the level of butyrate producing bacteria in the sample from the subject to the level of butyrate producing bacteria in a control sample; and (d) detecting that the level of butyrate producing bacteria in the sample from the subject is reduced or altered compared to the level of butyrate producing bacteria in the control sample, wherein the reduced or altered level of butyrate producing bacteria is indicative of the subject having colorectal cancer, non-dysplastic serrated polyps, or serrated crypt foci, or having a predisposition to colorectal cancer, non-dysplastic serrated polyps, or serrated crypt foci.

In some embodiments, the level of butyrate producing bacteria is the level of bacteria belonging to clostridial cluster XIVa or clostridial cluster IV. In some embodiments, the level of butyrate producing bacteria is the level of bacteria belonging to the phylum Firmicutes, the phylum Bacteroidetes, or a combination thereof. In some embodiments, the level of butyrate producing bacteria is the level of bacteria belonging to the genus Roseburia, the genus Eubacterium, the genus Faecalibacterium, the genus Clostridium, the genus Prevotella, or a combination thereof. In some embodiments, the level of butyrate producing bacteria is the level of Roseburia hominis, Roseburia faecis, Eubacterium rectale, Eubacterium hallii, Faecalibacterium prausnitzii, Clostridium sp. str., M62/1, Clostridium sp. SS2/1, or a combination thereof. In some embodiments, the level of butyrate producing bacteria is the level of mucin-degrading bacteria.

The level of butyrate producing bacteria can be measured by any techniques known in the art including but not limited to, bacterial 16S rRNA gene sequencing, bacterial 16S rRNA qPCR, RT-PCR, qRT-PCR, and whole genome sequencing. Samples can include but are not limited to feces and a tissue biopsy. In some embodiments, the level of bacteria is measured by the number of copies of bacterial 16S rRNA gene per gram of feces. In some embodiments, the cecal levels of short chain fatty acids are measured the sample. In some embodiments, the control sample is from a subject not suffering from colorectal cancer, non-dysplastic serrated polyps, or serrated crypt foci. In a further embodiment, the control sample is from a subject not suffering from CIMP colorectal cancer. In another embodiment, the control sample is not cancer cells. In some embodiments, the method further comprises administering butyrate producing bacteria, dietary fiber, sodium butyrate, or a combination thereof to the subject if the subject is diagnosed with colorectal cancer, non-dysplastic serrated polyps, or serrated crypt foci, and/or if the level of butyrate producing bacteria is altered or reduced.

In certain aspects, the invention provides a method of detecting, determining or diagnosing colorectal cancer, non-dysplastic serrated polyps, or serrated crypt foci in a subject comprising: (a) obtaining a sample from a subject; (b) measuring the alpha diversity of the bacteria in the sample from the subject; (c) comparing the level of alpha diversity in the sample from the subject to the level of alpha diversity in a control sample; and (d) detecting that the level of alpha diversity in the sample from the subject is reduced or altered compared to the level of alpha diversity in the control sample, wherein the reduced or altered level of alpha diversity is indicative of the subject having colorectal cancer, non-dysplastic serrated polyps, or serrated crypt foci, or having a predisposition to colorectal cancer, non-dysplastic serrated polyps, or serrated crypt foci.

The alpha diversity in the sample can be measured by any method known in the art including but not limited to bacterial 16S rRNA gene sequencing, alternatively combined with an algorithm. Samples can include but are not limited to feces and a tissue biopsy. In some embodiments, the cecal levels of short chain fatty acids are measured the sample. In some embodiments, the control sample is from a subject not suffering from colorectal cancer, non-dysplastic serrated polyps, or serrated crypt foci. In a further embodiment, the control sample is from a subject not suffering from CIMP colorectal cancer. In another embodiment, the control sample is not cancer cells. In some embodiments, the method further comprises administering butyrate producing bacteria, dietary fiber, sodium butyrate, or a combination thereof to the subject if the subject is diagnosed with colorectal cancer, non-dysplastic serrated polyps, or serrated crypt foci, and/or if the level of alpha diversity is altered or reduced.

In certain aspects, the invention provides a method of detecting, determining or diagnosing colorectal cancer, non-dysplastic serrated polyps, or serrated crypt foci in a subject comprising: (a) obtaining a sample from a subject; (b) measuring the operational taxonomic units (OTUs) in the sample from the subject; (c) comparing the level of the operational taxonomic units (OTUs) in the sample from the subject the level of the operational taxonomic units (OTUs) in a control sample; and (d) detecting that the level of the operational taxonomic units (OTUs) in the sample from the subject is reduced or altered compared to the level of the operational taxonomic units (OTUs) in the control sample, wherein the reduced or altered level of the operational taxonomic units (OTUs) is indicative of the subject having colorectal cancer, non-dysplastic serrated polyps, or serrated crypt foci, or having a predisposition to colorectal cancer, non-dysplastic serrated polyps, or serrated crypt foci.

The OTUs in the sample can be measured by any method known in the art including but not limited to bacterial 16S rRNA gene sequencing, alternatively combined with an algorithm. Samples can include but are not limited to feces and a tissue biopsy. In some embodiments, the cecal levels of short chain fatty acids are measured the sample. In some embodiments, the control sample is from a subject not suffering from colorectal cancer, non-dysplastic serrated polyps, or serrated crypt foci. In a further embodiment, the control sample is from a subject not suffering from CIMP colorectal cancer. In another embodiment, the control sample is not cancer cells. In some embodiments, the method further comprises administering butyrate producing bacteria, dietary fiber, sodium butyrate, or a combination thereof to the subject if the subject is diagnosed with colon cancer, non-dysplastic serrated polyps, or serrated crypt foci, and/or the OTUs are altered or reduced.

In certain aspects, the invention provides a method of detecting, determining or diagnosing colorectal cancer, non-dysplastic serrated polyps, or serrated crypt foci in a subject comprising: (a) obtaining a sample from a subject; (b) measuring the level of bacteria belonging to the order Lactobacillales in the sample from the subject; (c) comparing the level of bacteria belonging to the order Lactobacillales in the sample from the subject the level of bacteria belonging to the order Lactobacillales in a control sample; and (d) detecting that the level of bacteria belonging to the order Lactobacillales in the sample from the subject is increased compared to the level of bacteria belonging to the order Lactobacillales in the control sample, wherein the increased level of bacteria belonging to the order Lactobacillales is indicative of the subject having colorectal cancer, non-dysplastic serrated polyps, or serrated crypt foci, or having a predisposition to colorectal cancer, non-dysplastic serrated polyps, or serrated crypt foci.

The level of Lactobacillales bacteria can be measured by any techniques known in the art including but not limited to, bacterial 16S rRNA gene sequencing, bacterial 16S rRNA qPCR, RT-PCR, qRT-PCR, and whole genome sequencing. Samples can include but are not limited to feces and a tissue biopsy. In some embodiments, the level of bacteria is measured by the number of copies of bacterial 16S rRNA gene per gram of feces. In some embodiments, the cecal levels of short chain fatty acids are measured the sample. In some embodiments, the control sample is from a subject not suffering from colorectal cancer, non-dysplastic serrated polyps, or serrated crypt foci. In a further embodiment, the control sample is from a subject not suffering from CIMP colorectal cancer. In another embodiment, the control sample is not cancer cells. In some embodiments, the method further comprises administering butyrate producing bacteria, dietary fiber, sodium butyrate, or a combination thereof to the subject if the subject is diagnosed with colorectal cancer, non-dysplastic serrated polyps, or serrated crypt foci, and/or if the level of Lactobacillales bacteria butyrate producing bacteria is altered or increased.

In some embodiments, the cecal levels of short chain fatty acids are measured in the sample. In certain aspects, the invention provides a method of detecting, determining or diagnosing colon cancer, non-dysplastic serrated polyps, or serrated crypt foci in a subject comprising: (a) obtaining a sample from a subject; (b) measuring the cecal levels of short chain fatty acids in the sample from the subject; (c) comparing the level of the cecal levels of short chain fatty acids in the sample from the subject the level of the cecal levels of short chain fatty acids in a control sample; and (d) detecting that the cecal levels of short chain fatty acids in the sample from the subject is reduced or altered compared to the cecal levels of short chain fatty acids in the control sample, wherein the reduced or altered cecal levels of short chain fatty acids is indicative of the subject having colorectal cancer, non-dysplastic serrated polyps, or serrated crypt foci, or having a predisposition to colorectal cancer, non-dysplastic serrated polyps, or serrated crypt foci.

The cecal levels of short chain fatty acids can be measured by any method known in the art including but not limited to electron ionization-GCMS. Samples can include but are not limited to feces and a tissue biopsy. In some embodiments, the control sample is from a subject not suffering from colorectal cancer, non-dysplastic serrated polyps, or serrated crypt foci. In a further embodiment, the control sample is from a subject not suffering from CIMP colorectal cancer. In another embodiment, the control sample is not cancer cells. In some embodiments, the short chain fatty acid is butyrate. In some embodiments, the method further comprises administering butyrate producing bacteria, dietary fiber, sodium butyrate or a combination thereof to the subject if the subject is diagnosed with colon cancer, non-dysplastic serrated polyps, or serrated crypt foci, and/or the level of short chain fatty acids is altered or reduced.

In certain aspects, the invention provides a method of detecting, determining or diagnosing colorectal cancer, non-dysplastic serrated polyps, or serrated crypt foci in a subject comprising: (a) obtaining a sample from a subject; (b) measuring the level of bacterial butyryl coenzyme A nucleic acid (CoA), bacterial acetate CoA transferase nucleic acid (BcoA), or a combination thereof, in the sample from the subject; (c) comparing the level of bacterial butyryl coenzyme A nucleic acid (CoA), bacterial acetate CoA transferase nucleic acid (BcoA), or a combination thereof, in the sample from the subject to the level of bacterial butyryl coenzyme A nucleic acid (CoA), bacterial acetate CoA transferase nucleic acid (BcoA), or a combination thereof in a control sample; and (d) detecting that the level of bacterial butyryl coenzyme A nucleic acid (CoA), bacterial acetate CoA transferase nucleic acid (BcoA), or a combination thereof in the sample from the subject is altered or reduced compared to the level of bacterial butyryl coenzyme A nucleic acid (CoA), bacterial acetate CoA transferase nucleic acid (BcoA), or a combination thereof in the control sample, wherein the altered or decreased level of bacterial butyryl coenzyme A nucleic acid (CoA), bacterial acetate CoA transferase nucleic acid (BcoA), or a combination thereof is indicative of the subject having colorectal cancer, non-dysplastic serrated polyps, or serrated crypt foci, or having a predisposition to colorectal cancer, non-dysplastic serrated polyps, or serrated crypt foci.

In some embodiments, the nucleic acid is genomic DNA, mRNA, or cDNA. In some embodiments, the BcoA is the BcoA gene from Faecalibacterium prausnitzii. In some embodiments, the BcoA is the BcoA gene from the genus Roseburia. In some embodiments, the BcoA is the BcoA gene from the genus Eubacterium. In some embodiments, the BcoA is the BcoA gene from Clostridium sp. SS2/1. The level of CoA or BcoA can be measured by any method known in the art including but not limited to qPCR, RT-PCR, and qRT-PCR. In some embodiments, the sample is feces. In some embodiments, the sample is a tissue biopsy. In some embodiments, the level of BcoA is measured by is the number of copies of the BcoA gene per gram of feces. In some embodiments, the cecal levels of short chain fatty acids are measured the sample. In some embodiments, the control sample is from a subject not suffering from colorectal cancer, non-dysplastic serrated polyps, or serrated crypt foci. In a further embodiment, the control sample is from a subject not suffering from CIMP colorectal cancer. In another embodiment, the control sample is not cancer cells. In some embodiments, the method further comprises administering butyrate producing bacteria, dietary fiber, sodium butyrate, or a combination thereof to the subject if the subject is diagnosed with colorectal cancer, non-dysplastic serrated polyps, or serrated crypt foci, and/or if the level of CoA or BcoA is altered or reduced.

In certain aspects, the invention provides a method of detecting, determining or diagnosing colorectal cancer, non-dysplastic serrated polyps, or serrated crypt foci in a subject comprising: (a) obtaining a sample from a subject; (b) contacting nucleic acid from the sample with at least one primer that specifically hybridizes to the 16S rRNA nucleotide sequence, the BcoA nucleotide sequence, or the CoA nucleotide sequence of at least one butyrate producing bacteria; (c) subjecting the nucleic acid and the primer to amplification conditions; and (c) measuring the level of amplification product, wherein if the level of amplification product in the sample from the subject is altered or reduced compared to the level of amplification product in a control sample is indicative of the subject having colorectal cancer, non-dysplastic serrated polyps, or serrated crypt foci, or having a predisposition to colorectal cancer, non-dysplastic serrated polyps, or serrated crypt foci.

In some embodiments, the nucleic acid from the biological sample is a cDNA. In some embodiments, the level of amplification product is measured using at least one probe the specifically hybridizes to the amplification product. In some embodiments, the butyrate producing bacteria is belongs to clostridial cluster XIVa or clostridial cluster IV. In some embodiments, the butyrate producing bacteria is belongs to the phylum Firmicutes, the phylum Bacteroidetes, or a combination thereof. In some embodiments, the butyrate producing bacteria is belongs to the genus Roseburia, the genus Eubacterium, the genus Faecalibacterium, the genus Clostridium, the genus Prevotella, or a combination thereof. In some embodiments, the butyrate producing bacteria is Roseburia hominis, Roseburia faecis, Eubacterium rectale, Eubacterium hallii, Faecalibacterium prausnitzii, Clostridium sp. str., M62/1, Clostridium sp. SS2/1, or combinations thereof. In some embodiments, the control sample is from a subject not suffering from colorectal cancer, non-dysplastic serrated polyps, or serrated crypt foci. In a further embodiment, the control sample is from a subject not suffering from CIMP colorectal cancer. In another embodiment, the control sample is not cancer cells. In some embodiments, the method further comprises administering butyrate producing bacteria, dietary fiber, or a combination thereof to the subject if the subject is diagnosed with colorectal cancer, non-dysplastic serrated polyps, or serrated crypt foci. In some embodiments, the method further comprises administering sodium butyrate to the subject.

The invention further provides for methods of monitoring the level of butyrate producing bacteria, wherein the current methods of detection, determination, and diagnosis of colorectal cancer or the risk of developing colorectal cancer is repeated more than one time. This is particularly beneficial and important for subjects at risk of developing colorectal cancer (e.g. CIMP colorectal cancer), non-dysplastic serrated polyps, or serrated crypt foci, or has colorectal cancer (e.g. CIMP colorectal cancer), non-dysplastic serrated polyps, or serrated crypt foci.

In any of the methods of the invention, the butyrate producing bacteria can be is measured by measuring the level of the 16S rRNA gene of the butyrate producing bacteria or by measuring the diversity of bacterial 16S rRNA genes in the sample. The 16S rRNA gene can be measured, detected and/or identified using 16S rRNA gene sequencing, qPCR, RT-PCR, or qRT-PCR. In some embodiments, 16S rRNA gene expression level is measured by qPCR, RT-PCR, or qRT-PCR. Butyrate producing bacteria can be also measured by any method known in the art including but not limited to whole genome sequencing. Butyrate producing bacteria can be also measured by measuring the level of CoA or BcoA gene expression of the butyrate producing bacteria. In some embodiments, the CoA or BcoA gene is measured, detected and/or identified by qPCR, RT-PCR, or qRT-PCR. In some embodiments, CoA or BcoA gene expression level is measured by qPCR, RT-PCR, or qRT-PCR.

In a further embodiment, the numbers of butyrate producing bacteria can be determined by detecting the expression of a bacterial 16S rRNA gene, a bacterial CoA gene, a bacterial BcoA gene, or a combination thereof. In some embodiments, the detecting comprises detecting in the sample whether there is a reduction in an mRNA or cDNA encoding a bacterial 16S rRNA gene, a bacterial CoA gene, a bacterial BcoA gene, or a combination thereof. In some embodiments, the detecting comprises detecting in the sample where there is a reduction in a bacterial 16S rRNA gene, a bacterial CoA gene, a bacterial BcoA gene, or a combination thereof, wherein the bacterial 16SrRNA gene, the bacterial CoA gene, or the bacterial BcoA gene is from at least one butyrate producing bacteria. The reduction in a bacterial 16S rRNA gene, a bacterial CoA gene, a bacterial BcoA gene, or a combination thereof, in the sample can be detected through the genotyping of a sample, for example via gene sequencing, selective hybridization, amplification, gene expression analysis, or a combination thereof. The sample can comprise blood, serum, ileum tissue, cecum tissue, muscle tissue, feces, or a combination thereof.

The reduction or alteration can be detected at the DNA, RNA or polypeptide level of the 16S rRNA gene, CoA gene or BcoA gene. The detection can done by performing an oligonucleotide ligation assay, a confirmation based assay, a hybridization assay, a sequencing assay, an allele-specific amplification assay, a microsequencing assay, a melting curve analysis, a denaturing high performance liquid chromatography (DHPLC) assay, or a combination thereof. In some embodiments, the detection is performed by sequencing all or part of a 16S rRNA gene, CoA gene or BcoA gene of at least one BPB, or by selective hybridization or amplification of all or part of a 16S rRNA gene, CoA gene or BcoA gene of at least one BPB. A 16S rRNA gene, CoA gene or BcoA gene specific amplification can be carried out before the alteration identification step. In some embodiments, the detection is performed by selective hybridization or amplification of all or part of a 16S rRNA, CoA mRNA or BcoA mRNA of at least one butyrate producing bacteria. 16S rRNA, CoA mRNA or BcoA mRNA can be reverse transcribed to cDNA and/or amplified before the identification step.

In another embodiment, the method can comprise detecting the presence of an altered RNA expression of a 16S rRNA gene, CoA gene or BcoA gene of at least one butyrate producing bacteria. Altered RNA expression includes the presence of an altered RNA sequence, the presence of an altered RNA splicing or processing, or the presence of an altered quantity of RNA. These can be detected by various techniques known in the art, including by sequencing all or part of the RNA of a 16S rRNA gene, CoA gene or BcoA gene of at least one butyrate producing bacteria, or by selective hybridization or selective amplification of all or part of the RNA. In some embodiments, the detection is performed by selective hybridization or amplification of all or part of a 16S rRNA, CoA mRNA or BcoA mRNA. 16S rRNA, CoA mRNA or BcoA mRNA can be reverse transcribed to cDNA and/or amplified before the identification step.

In a further embodiment, the method can comprise detecting the presence of an altered polypeptide expression of a CoA gene or BcoA gene of at least one butyrate producing bacteria. Altered polypeptide expression includes the presence of an altered polypeptide sequence, the presence of an altered quantity of a CoA gene or BcoA gene of a butyrate producing bacteria. These can be detected by various techniques known in the art, including by sequencing and/or binding to specific ligands (such as antibodies). In one embodiment, the sample is contacted with an antibody specific for a CoA or BcoA of a butyrate producing bacteria and the formation of an immune complex is subsequently detected. Various methods for detecting an immune complex can be used, such as ELISA, radioimmunoassays (RIA), immune-enzymatic assays (IEMA), and FACS.

Various techniques known in the art can be used to detect or quantify altered gene expression, RNA expression, or sequence, which include, but are not limited to, hybridization, sequencing, amplification, and/or binding to specific ligands (such as antibodies). Other suitable methods include allele-specific oligonucleotide (ASO), oligonucleotide ligation, allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, denaturing HLPC, melting curve analysis, heteroduplex analysis, RNase protection, chemical or enzymatic mismatch cleavage, ELISA, radio-immunoassays (RIA) and immuno-enzymatic assays (IEMA). Some of these approaches (such as SSCA and CGGE) are based on a change in electrophoretic mobility of the nucleic acids, as a result of the presence of an altered sequence. According to these techniques, the altered sequence is visualized by a shift in mobility on gels. The fragments can then be sequenced to confirm the alteration. Some other approaches are based on specific hybridization between nucleic acids from the subject and a probe specific for wild type or altered gene or RNA. The probe can be in suspension or immobilized on a substrate. The probe can be labeled to facilitate detection of hybrids. Some of these approaches are suited for assessing a polypeptide sequence or expression level, such as Northern blot, ELISA and RIA. These latter require the use of a ligand specific for the polypeptide, for example, the use of a specific antibody.

Sequencing.

Sequencing can be carried out using techniques well known in the art, using automatic sequencers. The sequencing can be performed on the complete gene or on specific domains thereof, such as those known or suspected to carry deleterious mutations or other alterations.

Amplification.

Amplification is based on the formation of specific hybrids between complementary nucleic acid sequences that serve to initiate nucleic acid reproduction. Amplification can be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Useful techniques in the art encompass real-time PCR or qPCR, allele-specific PCR, PCR-SSCP, reverse transcription PCR (RT-PCR) or real time RT-PCR (qRT-PCR). Amplification usually requires the use of specific nucleic acid primers, to initiate the reaction. For example, nucleic acid primers useful for amplifying sequences from the gene or locus of a 16S rRNA gene, CoA gene or BcoA gene of a butyrate producing bacteria are able to specifically hybridize with a portion of the gene locus that flanks a target region of the locus. In one embodiment, amplification comprises using forward and reverse primers and/or probes.

The invention provides for a nucleic acid primer, wherein the primer can be complementary to and hybridize specifically to a portion of a coding sequence (e.g., gene or RNA) of a 16S rRNA gene, CoA gene or BcoA gene of at least one butyrate producing bacteria. Primers of the invention can thus be specific for a gene or RNA of a 16S rRNA gene, CoA gene or BcoA gene of a butyrate producing bacteria. By using such primers, the detection of an amplification product indicates the presence of or expression of the gene or the absence of such gene. Examples of primers of this invention can be single-stranded nucleic acid molecules of about 5 to 60 nucleotides in length, or about 8 to about 25 nucleotides in length. The sequence can be derived directly from the sequence of a 16S rRNA gene, a CoA gene or a BcoA gene of a butyrate producing bacteria. Perfect complementarity is useful, to ensure high specificity. However, certain mismatch can be tolerated. For example, a nucleic acid primer or a pair of nucleic acid primers can be used in a method for detecting colorectal cancer (e.g. CIMP colon cancer), non-dysplastic serrated polyps, or serrated crypt foci in a subject.

Amplification methods include, e.g., polymerase chain reaction, PCR (PCR Protocols, A Guide to Methods and Applications, 1990, ed. Innis, Academic Press, N.Y., and PCR Strategies, 1995, ed. Innis, Academic Press, Inc., N.Y., ligase chain reaction (LCR) (see, e.g., Wu, 1989, Genomics 4:560; Landegren, 1988, Science 241:1077; Barringer, 1990, Gene 89:117); transcription amplification (see, e.g., Kwoh, 1989, Proc. Natl. Acad. Sci. USA 86:1173); and, self-sustained sequence replication (see, e.g., Guatelli, 1990, Proc. Natl. Acad. Sci. USA 87:1874); Q Beta replicase amplification (see, e.g., Smith, 1996, J. Clin. Microbiol. 35:1477-1491), automated Q-beta replicase amplification assay (see, e.g., Burg, 1996, Mol. Cell. Probes 10:257-271) and other RNA polymerase mediated techniques (e.g., NASBA, Cangene, Mississauga, Ontario); see also Berger, 1987, Methods Enzymol. 152:307-316, 1987; Sambrook; Ausubel; U.S. Pat. Nos. 4,683,195 and 4,683,202; Sooknanan, 1995, Biotechnology 13:563-564. All the references stated above are incorporated by reference in their entireties.

Reverse transcription polymerase chain reaction (RT-PCR) is one of many variants of polymerase chain reaction (PCR). In one embodiment, RT-PCR can be used to detect RNA expression, for example, 16S rRNA, CoA RNA or BcoA RNA. RT-PCR can be used to detect gene expression through creation of complementary DNA (cDNA) transcripts from RNA. The RNA of interest can be reverse transcribed into its DNA complement through the use of reverse transcriptase. Subsequently, the newly synthesized cDNA can be amplified using traditional PCR. In some embodiment, quantitative PCR can be incorporated into RT-PCR (qRT-PCR) to quantify RNA. Quantitative PCR (qPCR) can be used to quantitatively measure the amplification of DNA using labelled probes (e.g. fluorescently labeled probes) or DNA dyes. In one embodiment, the level of butyrate producing bacteria is measured by detecting hybridization of a nucleic acid probe to a 16S rRNA gene or cDNA, a BcoA gene or cDNA, or a CoA gene or cDNA.

Selective Hybridization.

Hybridization detection methods are based on the formation of specific hybrids between complementary nucleic acid sequences that serve to detect nucleic acid sequence alteration(s). A detection technique involves the use of a nucleic acid probe specific for a gene or RNA, followed by the detection of the presence of a hybrid. The probe can be in suspension or immobilized on a substrate or support (for example, as in nucleic acid array or chips technologies). The probe can be labeled to facilitate detection of hybrids. For example, a sample from the subject can be contacted with a nucleic acid probe specific for a 16S rRNA gene, CoA gene or BcoA gene of a butyrate producing bacteria, and the formation of a hybrid can be subsequently assessed. In one embodiment, the method comprises contacting simultaneously the sample with a set of probes that are specific, respectively, for the 16S rRNA gene, CoA gene or BcoA gene of a butyrate producing bacteria. Thus, it is possible to detect directly the presence a 16S rRNA gene, CoA gene or BcoA gene of at least one butyrate producing bacteria, or more than one, in the sample. Also, various samples from various subjects can be treated in parallel.

According to the invention, a probe can be a polynucleotide sequence which is complementary to, and specifically hybridizes with, a target portion of, a 16S rRNA gene or RNA, a CoA gene or RNA, or a BcoA gene or RNA of a butyrate producing bacteria. Useful probes are those that are complementary to the 16S rRNA gene or RNA, a CoA gene or RNA, or a BcoA gene or RNA of a butyrate producing bacteria. Probes can comprise single-stranded nucleic acids of between 8 to 1000 nucleotides in length, for instance between 10 and 800, between 15 and 700, or between 20 and 500. Longer probes can be used as well. A useful probe of the invention is a single stranded nucleic acid molecule of between 8 to 500 nucleotides in length, which can specifically hybridize to a region of a gene or RNA from a butyrate producing bacteria.

The sequence of the probes can be derived from the sequences of the 16S rRNA gene, a CoA gene, or a BcoA gene of a butyrate producing bacteria. Nucleotide substitutions can be performed, as well as chemical modifications of the probe. Such chemical modifications can be accomplished to increase the stability of hybrids (e.g., intercalating groups) or to label the probe. Some examples of labels include, without limitation, radioactivity, fluorescence, luminescence, and enzymatic labeling.

A guide to the hybridization of nucleic acids is found in for example in Sambrook and Ausubel.

Specific Ligand Binding.

As indicated herein, expression of a CoA gene or a BcoA gene of a butyrate producing bacteria can also be detected by screening for alteration(s) in corresponding polypeptide sequence or expression levels. Different types of ligands can be used, such as specific antibodies. In one embodiment, the sample is contacted with an antibody specific for a CoA or a BcoA polypeptide and the formation of an immune complex is subsequently determined. Various methods for detecting an immune complex can be used, such as ELISA, radioimmunoassays (RIA) and immuno-enzymatic assays (IEMA).

For example, an antibody can be a polyclonal antibody, a monoclonal antibody, as well as fragments or derivatives thereof having substantially the same antigen specificity. Fragments include Fab, Fab′2, or CDR regions. Derivatives include single-chain antibodies, humanized antibodies, or poly-functional antibodies. An antibody specific for a CoA or BcoA polypeptide can be an antibody that selectively binds a CoA or BcoA polypeptide, respectively, namely, an antibody raised against a CoA or BcoA of a butyrate producing bacteria or an epitope-containing fragment thereof. Although non-specific binding towards other antigens can occur, binding to the target polypeptide occurs with a higher affinity and can be reliably discriminated from non-specific binding. In one embodiment, the method comprises contacting a sample from the subject with an antibody specific for a CoA or BcoA polypeptide, and determining the presence of an immune complex. Optionally, the sample can be contacted to a support coated with antibody specific for the CoA or BcoA polypeptide. In one embodiment, the sample can be contacted simultaneously, or in parallel, or sequentially, with various antibodies specific for different forms of a CoA or BcoA polypeptide, such as a wild type and various altered forms thereof.

The diagnosis methods can be performed in vitro, ex vivo, or in vivo. These methods utilize a sample from the subject in order to assess the level of butyrate producing bacteria.

The sample can be any biological sample derived from a subject, which contains bacteria, bacterial nucleic acids or polypeptides. Examples of such samples include, but are not limited to, fluids, tissues, cell samples, organs, or tissue biopsies. Non-limiting examples of samples include blood, or feces. The sample can be collected according to conventional techniques and used directly for diagnosis or stored. The sample can be treated prior to performing the method, in order to render or improve availability of nucleic acids or polypeptides for testing. Treatments include, for instance, lysis (e.g., mechanical, physical, or chemical), and/or centrifugation. Also, the nucleic acids and/or polypeptides can be pre-purified or enriched by conventional techniques, and/or reduced in complexity. Nucleic acids and polypeptides can also be treated with enzymes or other chemical or physical treatments to produce fragments thereof. In one embodiment, the sample is contacted with reagents, such as probes, primers, or ligands, in order to assess the presence of butyrate producing bacteria. Contacting can be performed in any suitable device, such as a plate, tube, well, or glass. In specific embodiments, the contacting is performed on a substrate coated with the reagent, such as a nucleic acid array or a specific ligand array. The substrate can be a solid or semi-solid substrate such as any support comprising glass, plastic, nylon, paper, metal, or polymers. The substrate can be of various forms and sizes, such as a slide, a membrane, a bead, a column, or a gel. The contacting can be made under any condition suitable for a complex to be formed between the reagent and the nucleic acids or polypeptides of the sample.

Identifying a reduced level or reduced diversity of butyrate producing bacteria in the sample can be correlated to the presence, predisposition or stage of progression of colorectal cancer (e.g. CIMP colorectal cancer). For example, an individual having a reduced level of butyrate producing bacteria has an increased risk of developing colorectal cancer (e.g. CIMP colorectal cancer). The determination of the presence of a reduced level or reduced diversity of butyrate producing bacteria in a subject also allows the design of appropriate therapeutic intervention, which is more effective and customized. Also, this determination at the pre-symptomatic level allows a preventive regimen to be applied.

The invention also provides for a diagnostic kit comprising products and reagents for detecting in a sample from a subject the presence of a 16S rRNA gene, a CoA gene, or a BcoA gene of at least one butyrate producing bacteria, or CoA polypeptide or BcoA polypeptide; alteration in the expression of 16S rRNA gene, a CoA gene, or a BcoA gene of at least one butyrate producing bacteria, and/or a CoA or BcoA polypeptide. The kit can be useful for determining whether a sample from a subject exhibits a reduced number of butyrate producing bacteria. For example, the diagnostic kit according to the present invention can comprise any primer, any pair of primers, any nucleic acid probe, antibody, and/or any ligand. The diagnostic kit according to the present invention can further comprise reagents and/or protocols for performing a hybridization, amplification or antigen-antibody immune reaction. In one embodiment, the kit can comprise nucleic acid primers that specifically hybridize to and can prime a polymerase reaction from a 16S rRNA gene, a CoA gene, or a BcoA gene of a butyrate producing bacteria.

### Administration and Dosing

A butyrate producing bacteria, dietary fiber (e.g. inulin fiber), or a combination thereof, can be administered to the subject once (e.g., as a treatment). Alternatively, butyrate producing bacteria, dietary fiber (e.g. inulin fiber), or a combination thereof, of the invention can be administered once a day, twice a day, three times a day, four times a day, five times a day, up to six times a day, preferably at regular intervals, to a subject in need thereof for a period of from about two to about twenty-eight days, or from about seven to about ten days. It can also be administered once or twice daily to a subject for a period of 1, 2, 3, 4, 5, 6, 7, 8, 9, 10, 11, 12 times per year, or a combination thereof. Furthermore, the butyrate producing bacteria and/or dietary fiber (e.g. inulin fiber) of the invention can be co-administrated with another therapeutic, such as a chemotherapeutic drug. The butyrate producing bacteria and/or dietary fiber (e.g. inulin fiber) of the invention can also be co-administrated with sodium butyrate. Where a dosage regimen comprises multiple administrations, the effective amount of the butyrate producing bacteria and/or dietary fiber (e.g. inulin fiber) administered to the subject can comprise the total amount of treatment administered over the entire dosage regimen. A subject can be monitored for improvement during the therapy and the dosages adjusted.

The butyrate producing bacteria and/or dietary fiber (e.g. inulin fiber) of the invention can be administered to a subject by any means suitable for delivering the butyrate producing bacteria and/or dietary fiber (e.g. inulin fiber) to the subject. For example, butyrate producing bacteria and/or dietary fiber (e.g. inulin fiber) can be administered by oral ingestion or rectally. In addition, sodium butyrate can be administered by oral ingestion or rectally.

The compositions of this invention can be formulated and administered to reduce the symptoms associated with colorectal cancer, non-dysplastic serrated polyps, or serrated crypt foci by any means that produces contact of the active ingredient with the agent's site of action in the body of an animal. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Pharmaceutical compositions for use in accordance with the invention can be formulated in conventional manner using one or more physiologically acceptable carriers or excipients. The therapeutic compositions of the invention can be formulated for a variety of routes of administration. Techniques and formulations generally can be found in Remington's Pharmaceutical Sciences, Meade Publishing Co., Easton, Pa. (1985), the entire disclosure of which is herein incorporated by reference.

Pharmaceutical formulations of the invention can comprise butyrate producing bacteria and/or dietary fiber (e.g. inulin fiber), mixed with a pharmaceutically-acceptable carrier. The pharmaceutical formulations of the invention can also comprise the butyrate producing bacteria and/or dietary fiber (e.g. inulin fiber) of the invention which are encapsulated.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelators (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid pharmaceutical compositions of the invention, conventional nontoxic solid pharmaceutically-acceptable carriers can be used; for example, pharmaceutical grades of mannitol, lactose, starch, magnesium stearate, sodium saccharin, talcum, cellulose, glucose, sucrose, or magnesium carbonate.

Solid formulations can be used for enteral (oral) or rectal administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, or magnesium carbonate. For oral or rectal administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral or rectal administration. The carrier can be selected from various oils including those of petroleum, animal, vegetable or synthetic origin, e.g., peanut oil, soybean oil, mineral oil, or sesame oil. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, and ethanol.

For oral or rectal administration, the therapeutic compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral or rectal administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Suitable enteral administration routes for the present methods include oral or rectal delivery. For example, the butyrate producing bacteria and/or dietary fiber (e.g. inulin fiber) of the invention can be administered by oral or rectal delivery. For oral or rectal administration, the therapeutic compositions are formulated into conventional oral or rectal administration forms such as capsules, tablets, suppositories and tonics.

The dosage administered can be a therapeutically effective amount of the composition sufficient to result in an increase in the number of butyrate producing bacteria and/or the treatment of colorectal cancer (e.g. CIMP colon cancer), non-dysplastic serrated polyps, or serrated crypt foci in a subject, and can vary depending upon known factors such as the pharmacodynamic characteristics of the active ingredient and its mode and route of administration; age, sex, health and weight of the recipient; nature and extent of symptoms; kind of concurrent treatment, frequency of treatment and the effect desired.

A therapeutically effective dose of butyrate producing bacteria and/or dietary fiber (e.g. inulin fiber) can depend upon a number of factors known to those or ordinary skill in the art. The dose(s) of the butyrate producing bacteria and/or dietary fiber (e.g. inulin fiber) can vary, for example, depending upon the identity, size, and condition of the subject or sample being treated, further depending upon the route by which the composition is to be administered, if applicable, and the effect which the practitioner desires the butyrate producing bacteria and/or dietary fiber (e.g. inulin fiber) to have upon the subject. These amounts can be readily determined by a skilled artisan.

Inulin fiber can be administered at about 5-10 grams per day, or at about 10-14 grams per day, or at about 14-25 grams per day, up to about 40 grams per day.

### Probiotic Therapy

An aspect of the invention provides a method of treating colon cancer (e.g. CIMP colon cancer), non-dysplastic serrated polyps, or serrated crypt foci by increasing the level of butyrate producing bacteria in the intestinal tract of a subject. In one embodiment, the butyrate producing bacteria of the invention are administered as a probiotic formulation or a food supplement. A mixture of butyrate producing bacteria may be present in the probiotic formulation or food supplement at a concentration from about 20 to about 60 weight percent. A mixture of butyrate producing bacteria may be present in the probiotic formulation or food supplement at a concentration from about 10 to about 50 weight percent. A mixture of butyrate producing bacteria may be present in the probiotic formulation or food supplement at a concentration from about 10 to about 99 weight percent.

The probiotic formulation or food supplement of the invention can take the various forms as prepared by conventional means with pharmaceutically acceptable excipients. Additional components including, but not limited to, dietary fiber (e.g. inulin fiber) can be present in the inventive probiotic formulation. Other soluble fibers (e.g. fructooligosaccharides) can also be present in the inventive probiotic formulation. The various ingredients of the probiotic formulation may be mixed together by conventional methods and formed into tablets for oral administration. Alternatively, the ingredients may be mixed together and placed into gelatin capsules. The inventive probiotic formulation may also contain conventional food supplement fillers and extenders such as, for example, rice flour. The dosage rate, effective as a food supplement and for reestablishing butyrate producing bacteria in the intestinal tract, can be readily determined by a skilled artisan.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Human Studies of the Role of the Microbiome in CIMP Colorectal Cancer

### Materials and Methods

### Subjects:

The microbiota of matched tumor and normal tissue from 5 individuals with a CIMP tumor (5 CIMP tumors and 5 CIMP normal tissue biopsies) compared to 5 individuals with a Non-CIMP tumor (5 Non-CIMP tumors and 5 Non-CIMP normal tissue biopsies) obtained from the Weill-Cornell GI Cancer Biobank were examined. In the operating room, CRC and adjacent healthy normal margin mucosal tissue were immediately placed into individual sterile cryovials and flash frozen in OCT as previously described for microbiome studies (Zhang, et al., 2005). Samples were well matched for subject age and tumor location to avoid confounding effects of these variables on the microbiota. None of the patients had taken any antibiotics in the 2 months prior to biopsy, and none had undergone chemotherapy, both of which can impact the microbiota. The day prior to the surgery, each study participant (CIMP and NON-CIMP groups) has the same bowel prep regimen, Golytely, and fasted the day of surgery.

### Nucleic Acid (NA) Extraction:

NA was extracted from needle biopsies (3-4 per tissue) from matched tumor and normal tissue biopsies from the patients. Both RNA and DNA were obtained using the Qiagen AllPrep RNA/DNA extraction kit, including bead-beating steps to promote bacterial cell wall disruption.

16S rRNA Gene Sequencing:

Individual samples from CIMP and Non-CIMP groups were amplified in triplicate with composite, Golay barcoded primers targeting the V4 variable regions of the bacterial 16S rRNA gene to generate libraries for Illumina MiSeq sequencing according to the Earth Microbiome Project standard protocol following the recommendations of Caporaso, et al. 2012. Triplicate barcoded V4 PCR products were combined, run on agarose gels, quantified (Picogreen), pooled (1 pool with all 200 human samples represented, 240 ng of DNA per sample) and cleaned (with the MoBio UltraClean PCR Clean-UP kit) as previously described (Caporaso, et al. 2012). PhiX was combined with the 16S library (30-50% PhiX) to enhance sequence complexity, and the library was sequenced on the Illumina MiSeq platform at the Center for Infection and Immunity using the 2×250 chemistry. 16S rRNA gene sequence analysis was performed using QIIME (Quantitative Insights Into Microbial Ecology) (Caporaso, et al. 2012; Kuczynski et al. 2012) according to the Illumina demultiplexing and processing protocol and quality-filtering recommendations (Edgar, et al. 2010; Lozupone, et al. 2013). Open-reference OTU picking (97% OTUs) was carried out in QIIME as newly recommended by the Earth Microbiome Project. Taxonomies were assigned using the RDP Classifier program trained on the most recent version of the Greengenes dataset. Alpha diversity (Shannon diversity, PD whole tree, chaol, and observed species) and beta diversity (weighted and unweighted UniFrac) metrics was assessed and visualized with rarefaction plots, principal coordinate analysis, hierarchical (UPGMA) clustering through jackknifed subsampling, and distance histograms. Linear discriminant analysis effect size (LEfSe) analysis was used to identify taxa that differ significantly between CIMP and Non-CIMP tissues (Segata, et al. 2011). LEfSe detects significant differences in abundance between groups (non-parametric factorial Kruskal-Wallis sum rank test), investigates biological consistency among subclasses (unpaired Wilcoxon rank-sum test) and estimates the effect size of differentially abundant organisms in 16S rRNA gene datasets (Segata, et al. 2011). For assessment of significant changes between paired tumor and normal tissue, the Wilcoxon signed-rank test was applied as previously described for the discovery of increased Fusobacterium spp. in tumor tissues from CRCs (Kostic, et al. 2012).

### Composition of the Bacterial Metagenome:

The functional composition of the bacterial metagenome was predicted and compared between CIMP and Non-CIMP biopsies with PICRUSt (phylogenetic investigation of communities by reconstruction of unobserved states) analysis. PICRUSt is a computational method for predicting the gene content and associated functional profile of the microbiome based on 16S rRNA gene sequencing data (Langille, et al. 2013). Phylogenies based on the 16S gene have been shown to recapitulate the phylogenies based on shared gene content obtained with shotgun sequencing (Segata and Huttenhower 2011; Snel, et al. 1999; Zaneveld, et al. 2010; Konstantinidis and Tieje 2005), indicating that, in many cases, microbial functions are predictable from marker gene information. Therefore, PICRUSt can be implemented, which uses an extended ancestral state reconstruction algorithm to estimate the gene contents of all organisms in the Greengenes 16S rDNA phylogeny and predicts the functional composition of a microbial community with an accuracy of greater than 80% (Langille, et al. 2013).

### Results

Pyrosequencing yielded 600,000 reads after initial quality filtering, corresponding to between 17,751 and 38,941 sequences per tissue biopsy (average sequence length was 447 bases).

### Alpha Diversity:

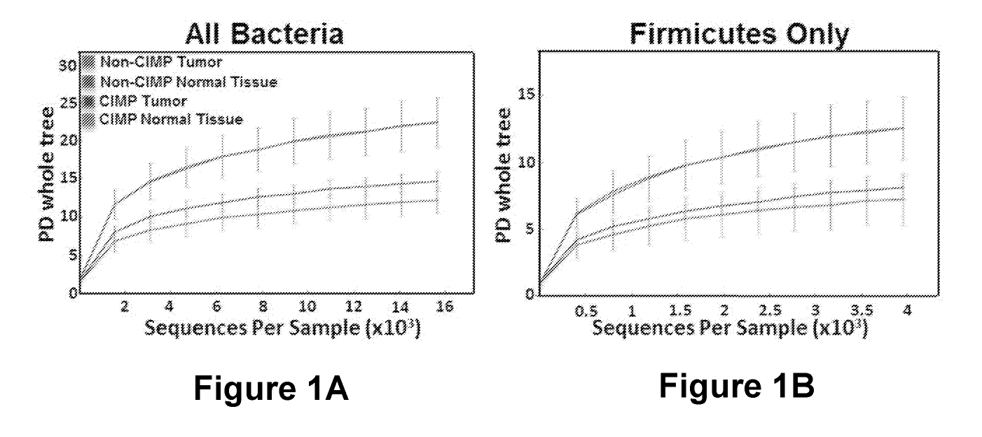

Alpha diversity in each sample was determined based on the entire microbiota (All Bacteria) using the QIIME pipeline for measures of phylogenetic diversity (PD) whole tree, chaol, observed species, and Shannon diversity index at a rarefaction depth of 15,650 sequences. The resulting rarefaction curves (PD whole tree shown in FIG. 1A) indicated a remarkable and highly significant decrease in all alpha diversity measures for CIMP normal tissue versus Non-CIMP normal tissue, as well as CIMP tumor tissue versus Non-CIMP tumor tissue. Significant differences in alpha diversity between groups were observed for all alpha diversity measures.

These results indicated that the overall bacterial species diversity and richness were significantly lower in CIMP tissues compared to Non-CIMP tissues. This reduction in diversity in CIMP was not only restricted to tumor tissue, but was apparent in unaffected normal tissue as well. This suggested an overall difference in the microbial biodiversity in individuals with CIMP tumors compared to individuals with Non-CIMP tumors. Significant differences were not observed when comparing CIMP tumors versus CIMP normal tissue or when comparing Non-CIMP tumors versus Non-CIMP normal tissue. Thus the major difference in diversity was found only between the two molecular phenotypes of CRC and was tissue-type independent. It was also found that the majority of the difference in diversity was attributable to reduced diversity of members of the phylum Firmicutes in CIMP patient tissues compared to Non-CIMP patient tissues (FIG. 1B).

### Beta Diversity:

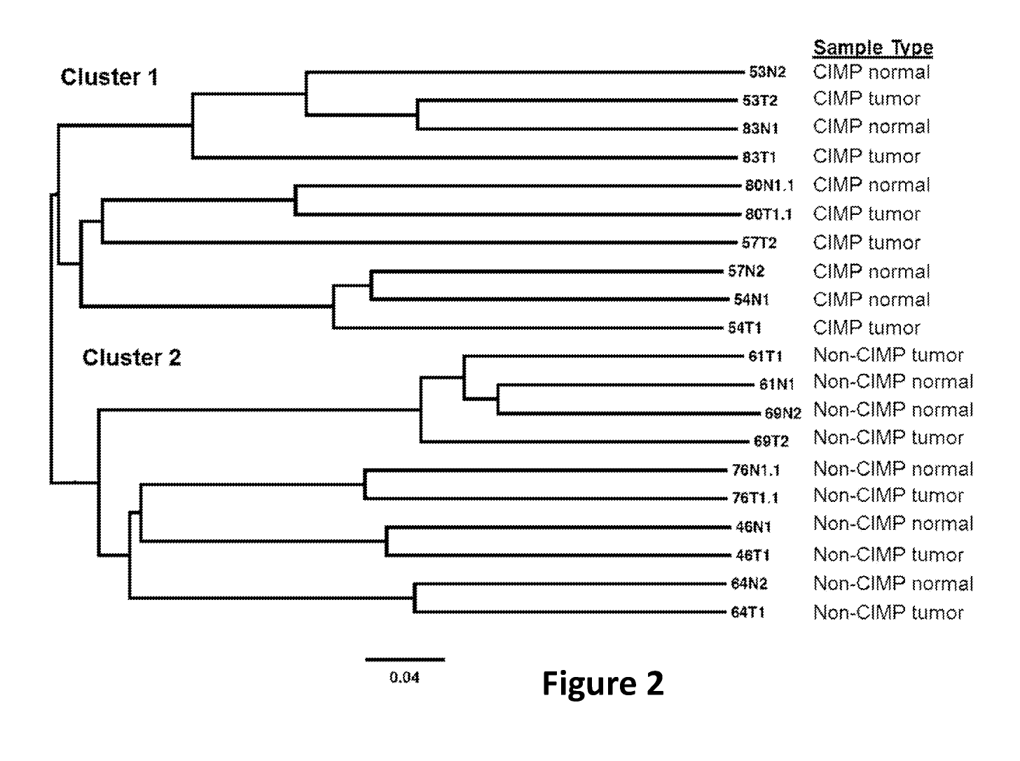

Beta diversity, such as the unweighted UniFrac metric, can determine whether bacterial communities are significantly different based on the lineages they contain (Lozupone and Knight 2005). A UPGMA tree showing the clustering of patient samples based on the unweighted UniFrac metric is shown in FIG. 2. This tree revealed two primary clusters based on all 20 tissue samples (5 CIMP tumor with 5 CIMP normal, 5 Non-CIMP tumor with 5 Non-CIMP normal). Remarkably, all CIMP tissues (normal and tumor) fell within Cluster 1, while all Non-CIMP tissues (normal and tumor) fell within Cluster 2. Within these clusters, subclusters were apparent. Interestingly, within CIMP tumors, the subclusters correlated with MSI status (data not shown) and within the Non-CIMP tumors, the subclusters correlated with KRAS exon 12 mutations (data not shown).

### Enriched OTUs:

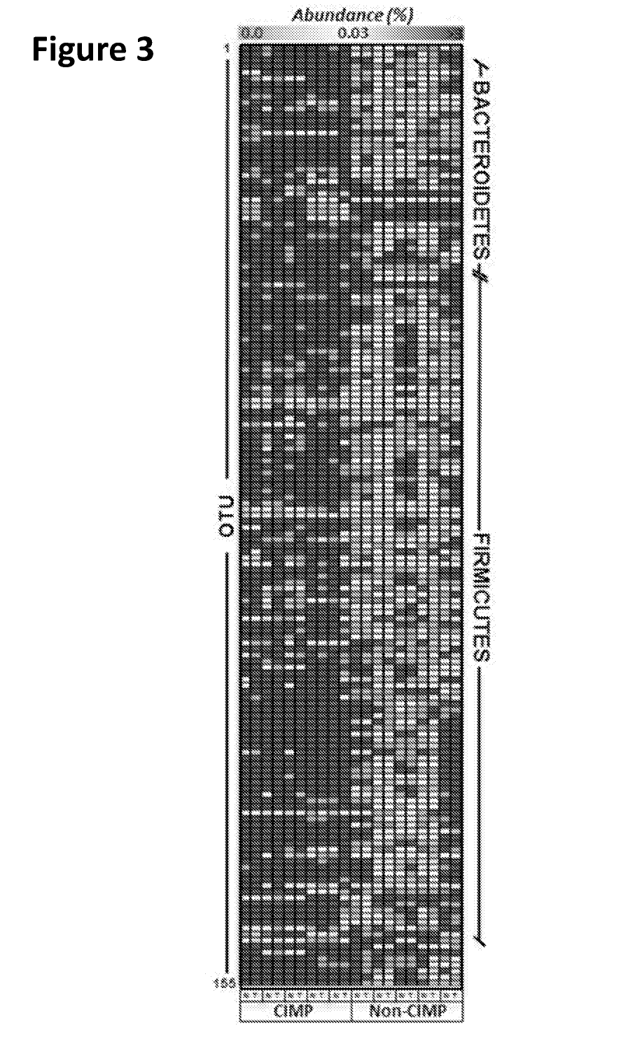

Operational taxonomic unit (OTU) clustering based on 97% similarity, which approximates species level OTUs, resulted in a total of 2526 OTUs in the total dataset. Given that the primary difference identified based on alpha and beta diversity was confirmed to be between CIMP tissues and Non-CIMP tissues (independent of tissue source; tumor or normal), a search for OTUs that differed significantly between CIMP and Non-CIMP tissue, independent of tissue type or sample matching was performed. This analysis revealed 155 OTUs that differed between the two groups (CIMP vs. Non-CIMP) (FIG. 3). Only 12 OTUs (7.7%) were enriched in CIMP tissues as compared to 143 (92.3%) enriched in Non-CIMP tissues. This was consistent with the reduced diversity observed in CIMP tissues compared to Non-CIMP tissues (see alpha diversity). The majority of OTUs that were depleted in the CIMP group were in the phylum Firmicutes (112/155 total OTUs; 72%) followed by the phylum Bacteroidetes (37/155 total OTUs; 24%).

Next it was determined which individual species contributed to these differences. Representative sequences from each of the 155 OTUs were blasted against the Greengenes database. Species-level determination was limited to those sequences with similarity matches greater than 96.5%. From the original 155 OTUs, 62 could be classified to the species level (39 Firmicutes, 18 Bacteroidetes, 3 Fusobacteria, and 2 Verrucomicrobia). Of the 39 species of Firmicutes, 27 species (69%) were from clostridial clusters XIVa and IV, which contain the majority of intestinal BPB.

### BPB Deficiency in CIMP Tissues Compared to Non-CIMP Tissues:

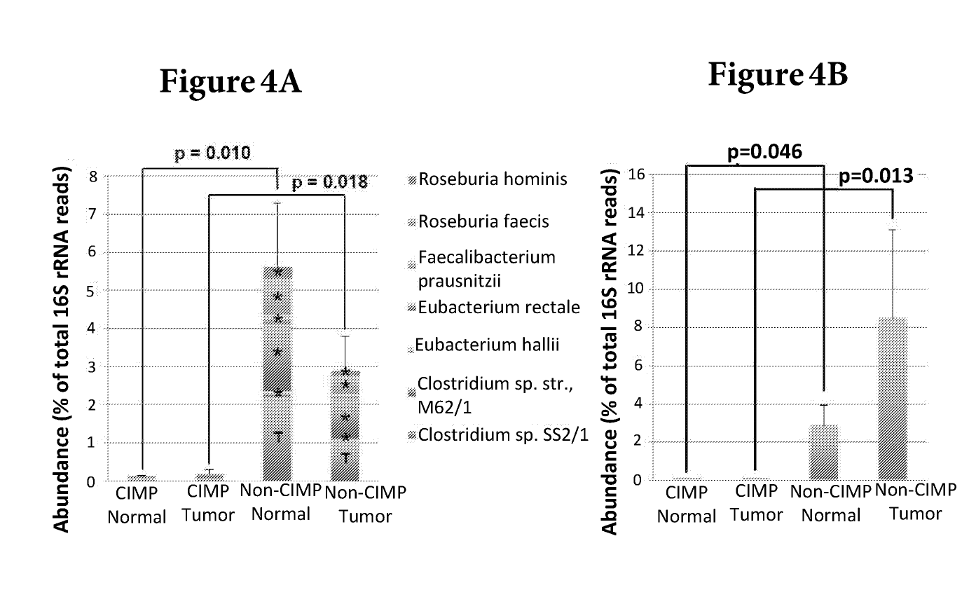

Within the clostridial clusters XIVa and IV, the major BPB that use the BcoA pathway have been described (Louis and Flint 2007; Louis and Flint 2009; Barcenilla, et al. 2000; Vital, et al. 2013). Seven of these BPB were significantly depleted in CIMP tissues compared to Non-CIMP tissues (Roseburia hominis, Roseburia faecis, Faecalibacterium prausnitzii, Eubacterium rectale, Eubacterium hallii, Clostridium sp. str., M62/1 and Clostridium sp. SS2/1). Stratification of the data by tumor type and tissue type (CIMP normal, CIMP tumor, Non-CIMP normal, Non-CIMP tumor) revealed significantly lower levels of these primary butyrate producers between CIMP normal tissue versus Non-CIMP normal tissue (Mann-Whitney, p=0.010), as well as between CIMP tumor tissue versus Non-CIMP tumor tissue (Mann-Whitney, p=0.018) (FIG. 4A). Furthermore, nearly all of these BPB were significantly lower in CIMP tissues compared to Non-CIMP tissues in this stratified comparison (asterisks in FIG. 4A).

### Prevotella Deficiency in CIMP Tissues Compared to Non-CIMP Tissues:

Reduced diversity of Bacteroidetes was associated with decreased Prevotella in CIMP tissues compared to Non-CIMP tissues. Stratification of data by tumor and tissue type revealed significantly lower levels of Prevotella between CIMP normal tissue versus Non-CIMP normal tissue (Mann-Whitney, p=0.046), as well as between CIMP tumor tissue versus Non-CIMP tumor tissue (Mann-Whitney, p=0.013) (FIG. 4B). This was consistent with a potential impact from dietary differences, as Prevotella is associated with plant fiber-based diets (DeFilippo, et al. 2010).

### Other Differences in Bacteria Level Between CIMP Tumor and Non Tumor Tissue:

Levels of mucin-degrading bacteria were also decreased, and Lactobacillales was increased in CIMP tissue. Additionally, the previous findings that tumor tissue is enriched in Fusobacteria was confirmed (Castellarin, et al. 2012; McCoy, et al. 2013) and Campylobacter sp. was also enriched in tumor tissue. Data not shown.

In conjunction with LEfSe (Segata, et al. 2011), PICRUSt analysis identified genes of the central operon of clostridial butyrate synthesis (i.e. thiolase, crotonase, β-hydroxybutyryl-CoA dehydrogenase, and butyryl-CoA dehydrogenase) (Louis, et al. 2007; Asanuma, et al. 2011), along with the BcoA gene and butyrate synthesis at the functional level, as being significantly deficient in CIMP compared to Non-CIMP patients (data not shown).

These results showed that butyrate production was not only reduced in the tumor tissues of CIMP patients, but also in the normal tissue of CIMP patients. Thus, the overall colonic microbiota is deficient in BPB in CIMP patients.

### Example 2—Human Studies of the Role of Butyrate in CIMP Colorectal Cancer

### Materials and Methods

### Subjects:

Thirty-four (34) new biopsy specimens were obtained from the Weill Cornell biobank (9 CIMP patients (matched tumor and normal tissue) and 8 Non-CIMP patients (matched tumor and normal tissue)). Samples were selected based on the same inclusion criteria described for samples used in Example 1 analyses.

Concentrations of short and medium chain fatty acids (butyrate, acetate, propionate, isovalerate, valerate, hexanoate, heptanoate, and octanoate) were measured in all 34 biopsies by the Michigan Regional Comprehensive Metabolomics Resource Core using electron ionization-GCMS.

### Results

### Butyrate was Deficient in CIMP Tissues Compared to Non-CIMP Tissues:

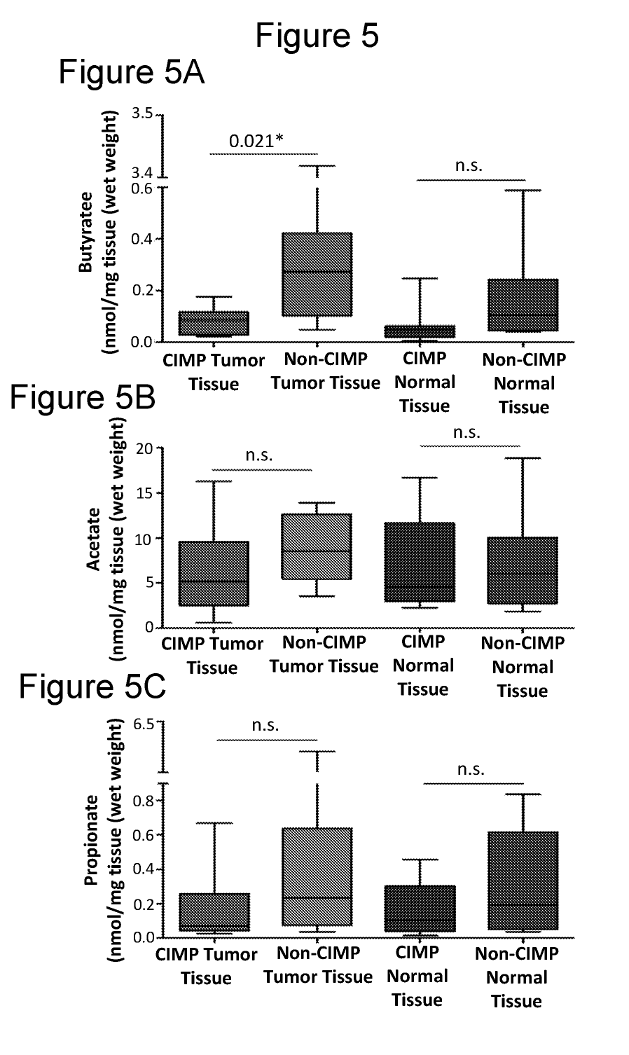

This analysis demonstrated significantly lower concentrations of butyrate in CIMP tumor tissue compared to Non-CIMP tumor tissue (Mann-Whitney, p=0.021), corresponding to an average 8-fold reduction in butyrate in CIMP tumors (FIG. 5A). In normal tissue from CIMP patients, there was an average 3-fold lower concentration of butyrate compared to normal tissue from Non-CIMP patients. In contrast, differences in concentrations of other SCFAs (acetate shown in FIG. 5B and propionate shown in FIG. 5C) and MCFAs (data not shown) did not differentiate tumor phenotypes. These results demonstrated a specific deficiency in tissue butyrate in patients with CIMP tumors.

### Example 3-Mouse Studies of the Role of the Microbiome and Butyrate in CIMP Colorectal Cancer

### Materials and Methods

### HBUS Mouse Model:

HBUS mice are a mouse model of serrated polyps. This double-transgenic mouse model expresses the EGFR ligand HB-EGF and a G protein-coupled receptor that facilitates processing of HB-EGF from the plasma membrane leading to increased phosphorylation of EGFR and downstream activation of the R-R-M-E-M pathway. HBUS mice develop macroscopic serrated polyps in the cecum that resemble human serrated hyperplastic polyps as early as 5 weeks. The cecal location of these serrated lesions situates them in a prime location for butyrate exposure from fermentation (Bongers, et al. 2012).

Differences in alpha diversity, beta diversity, and species-level OTU abundance in the microbiota of co-housed HBUS mice (n=20) and wild type mice (n=7) were analyzed as described in Example 1.

Additionally, in order to induce BPB deficiency in HBUS and wild type mice, mice were placed on a fiber-deficient diet and compared to HBUS mice on a high-fiber diet (supplemented with 6% inulin fiber) over a 9 week time course. BPB and BcoA were evaluated as described in Example 1 in fecal samples from wild type mice on the proposed no-fiber diet (D12329) (n=13) compared to mice on the same diet supplemented with 6% inulin (n=13) over a 9-week time course.

### Results

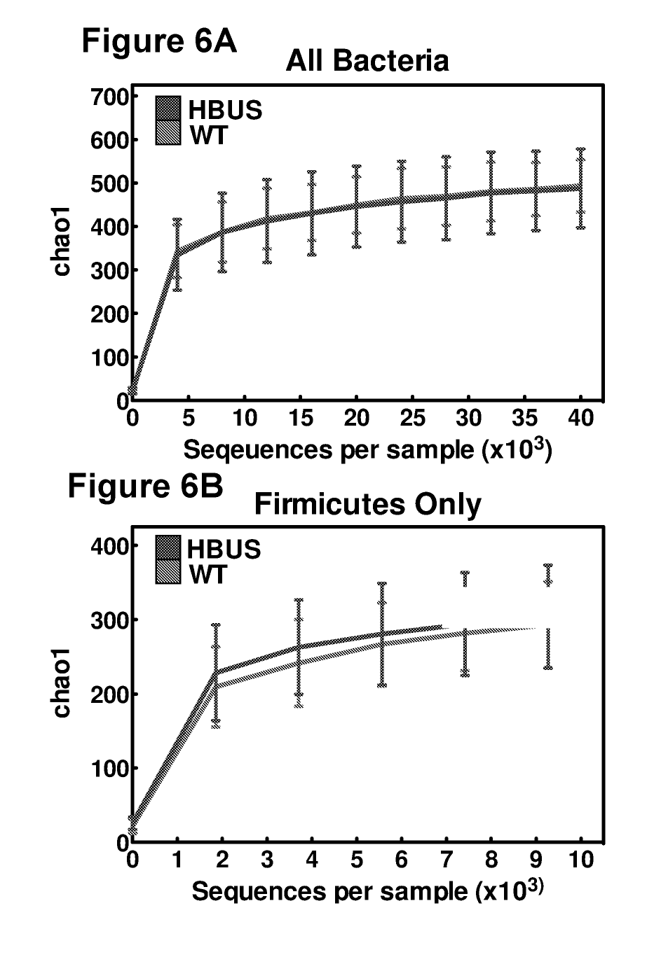

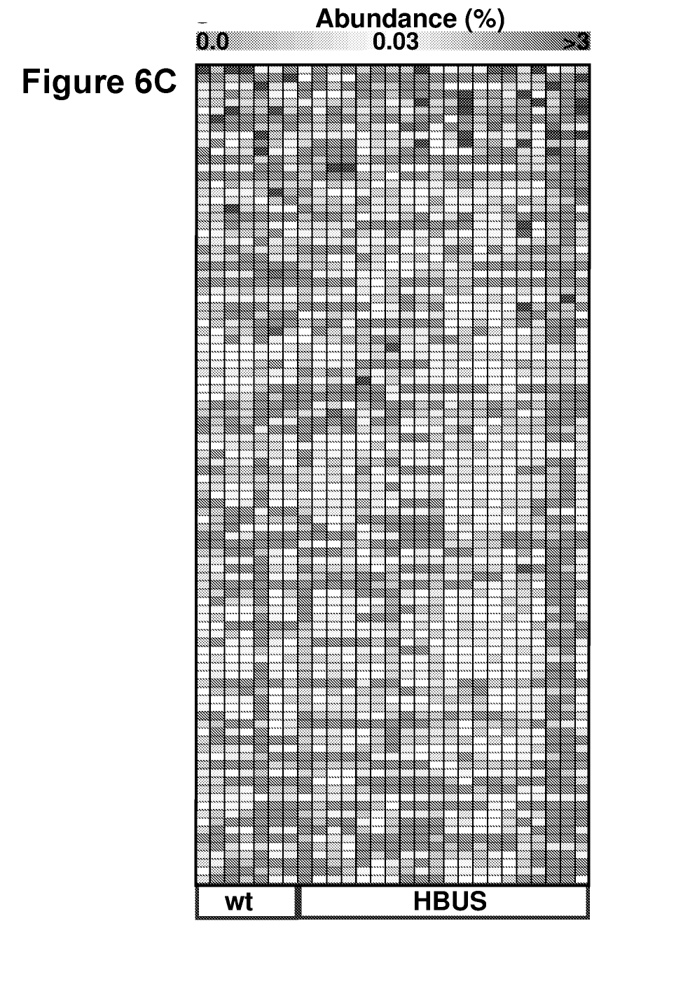

The analyses indicated that HBUS and wild type mice do not have differences in any of the alpha diversity metrics for total bacterial profiles (FIG. 6A) or the Firmicutes phylum (FIG. 6B). In addition, in co-housed HBUS and wild type mice clustered together based on hierarchical clustering of bacterial OTUs (Bongers, et al. 2012), there were no apparent differences in the abundance of OTUs between wild type and HBUS mice (FIG. 6C). Thus, while transgenic expression of the EGFR ligand HB-EGF and a G protein-coupled receptor in the HBUS model resulted in serrated polyp development, it did not substantially alter the microbiota. These congruent microbial profiles allowed the evaluation of the impact of diet-induced BPB deficiencies on tumorigenesis and CIMP in HBUS mice.

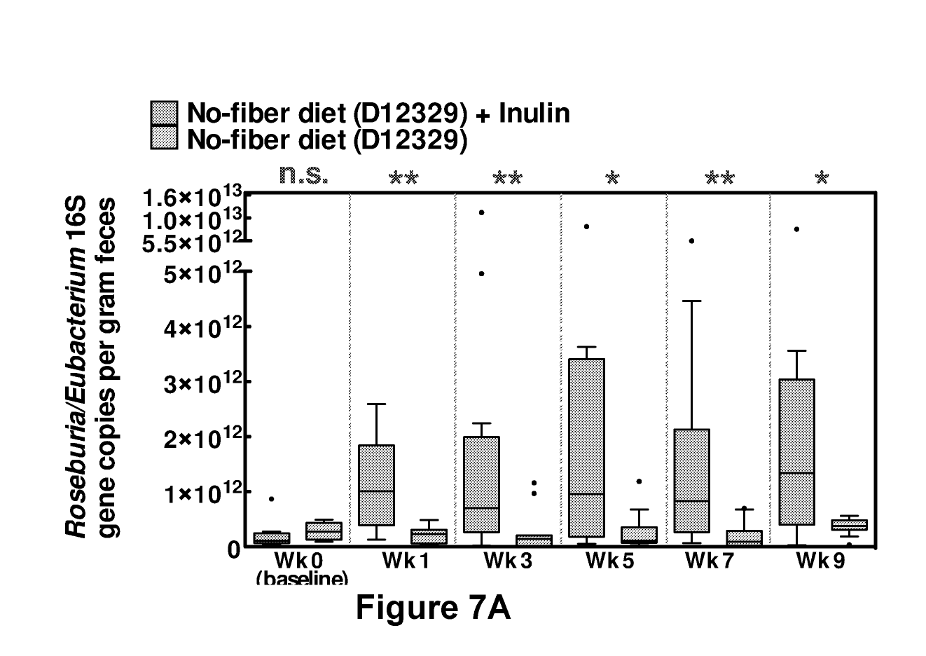

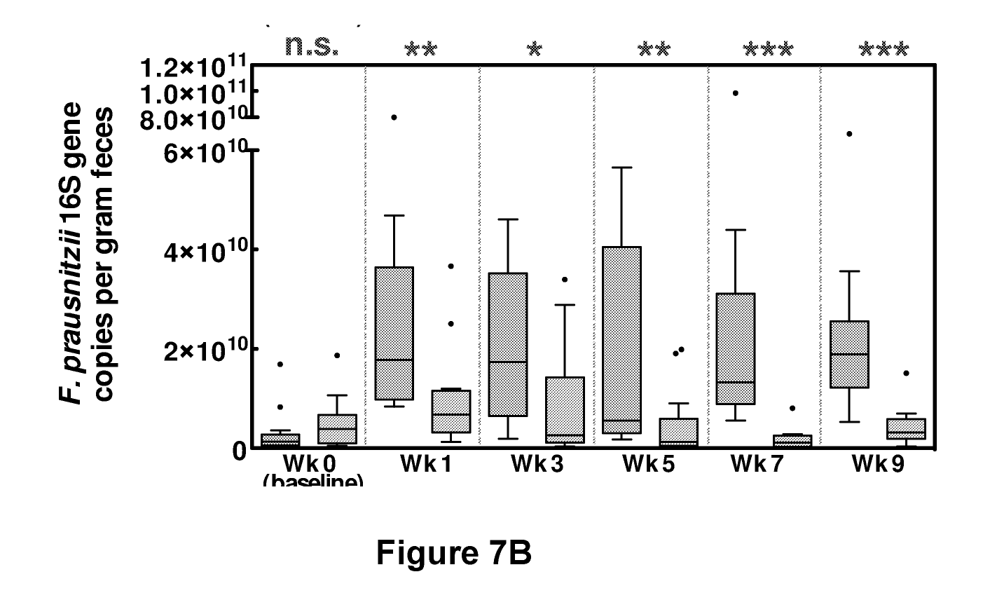

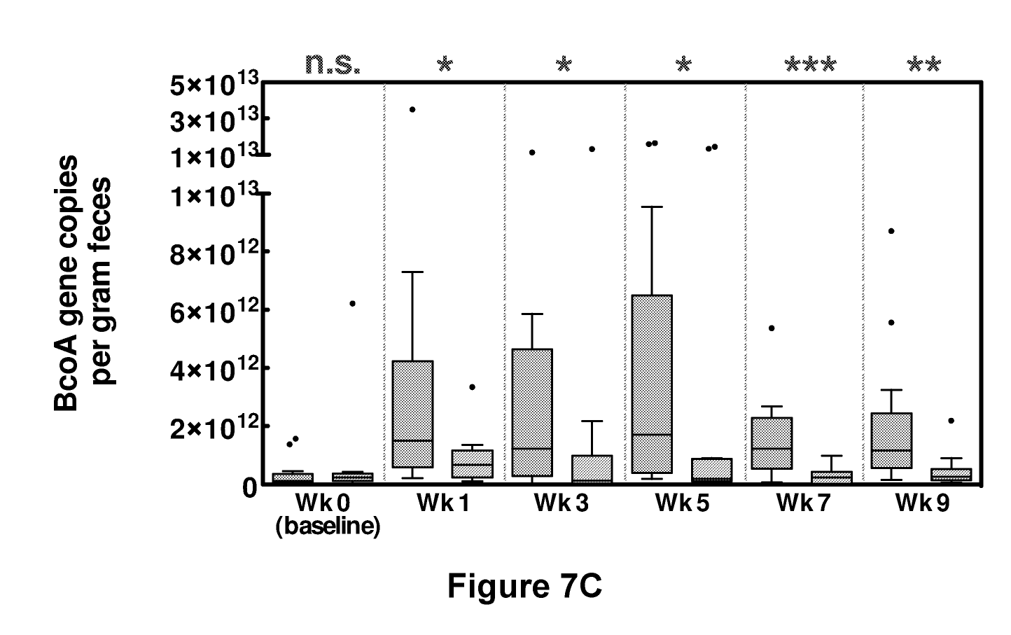

BPB and BcoA were evaluated in fecal samples from wild type mice on the proposed no-fiber diet (D12329) (n=13) compared to mice on the same diet supplemented with 6% inulin (n=13) (FIGS. 7A, 7B and 7C). BPB and BcoA gene quantities increased rapidly in mice on the high-fiber diet, being significantly elevated from baseline as early as one week after initiation of the diets (Roseburia/Eubacterium 16S, avg. fold increase=11.4; F. prausnitzii 16S, avg. fold increase=7.7; BcoA, avg. fold increase=8.7). In contrast, BPB and BcoA remained at weaning levels in mice on the no-fiber diet. At all time-points throughout the 9-week diet, levels of BPB and BcoA were significantly higher in mice on the high-fiber diet compared to mice on the no-fiber diet (FIGS. 7A-C, Mann-Whitney significance indicated by red asterisks). These findings suggested a rapid and sustained response of BPB to dietary fiber that will allow interrogation of the role of BPB in serrated tumorigenesis and CIMP.

### Example 4—Group-Specific Differences in the Microbiota Between CIMP and Non-CIMP Tumors

The data described herein in Example 1 indicated a significant difference in the microbiota of CIMP and Non-CIMP patients based on 16S rDNA pyrosequencing (FIGS. 1-4). BPB were reduced in both normal and tumor tissue from CIMP patients compared to Non-CIMP patients. In addition, the abundance of Prevotella spp. was decreased in CIMP tumors (FIG. 4B).

Group-specific differences in the microbiota are identified between matched tumor and normal tissue biopsies from 50 patients with CIMP tumors compared to 50 patients with Non-CIMP tumors using 16S rRNA gene high-throughput sequencing. Without being bound by theory, the intestinal microbiota differences between CIMP and Non-CIMP patients results in functional differences that influence tumorigenesis.

### Material and Methods

### Subjects:

Tissue specimens are obtained from the Weill Cornell Colorectal Cancer Biobank. Tissue specimens are restricted to non-familial colon cancer probands. A total of 200 samples (50 CIMP tumors and matched normal tissue and 50 Non-CIMP tumors and matched normal tissue) are evaluated. Tissue specimens from each patient consist of 3-4 needle biopsies from each tumor and normal tissue from the same colonic region. Clinical information is recorded by the Biobank for the following characteristics: gender, age at biopsy, anatomic tumor site, histological type, tumor grade, differentiation, recent medication use (including antibiotics), adjuvant therapy, pT designation, depth invasion, lymph node involvement, tumor size, small vessel invasion, extramural large vein invasion, tumor infiltrating lymphocytes, tumor budding, perineural invasion, and perforation. Tumor samples held by the Biobank have been screened for loss of MLH1, MSH2, MSH6, PMS2, MSS, MSI-H, MSI-L, and MLH1 methylation. ^{V600E}BRAF mutations, which are associated with CIMP, and KRAS mutations, which are mutually exclusive with ^{V600E}BRAF mutations, have also been screened. Thus, a complete molecular characterization of tumors for all patients is included in the study.

### Nucleic Acid (NA) Extraction:

NA is extracted as described in Example 1.

Validate CIMP and Non-CIMP Status in Tumor Specimens Using a Panel of Eight Methylation Markers with MethylLight Real-Time PCR:

Extracted DNA from tumor biopsies are treated with sodium bisulfite as previously described (Ogino, et al. 2006). Real-time PCR to measure DNA methylation (MethylLight) is performed on an ABI 7500 for quantitative real-time PCR. Eight CIMP-specific promoters will be interrogated: CACNA1G, CDKN2A (p16), CRABP1, IGF2, MLH1, NEUROG1, RUNX3, and SOCS1. COL2A1 are used to normalize the input of bisulfite-converted DNA. A PMR (percent of methylated reference) cutoff of 4 will be applied based on previous reports (Ogino, et al. 2006; Widschwendter, et al. 2004). CIMP-high tumors are defined by methylation of 6-8 of the CIMP-specific promoters; CIMP-low tumors are defined by methylation of 1-5 promoters as previously described (Ogino, et al. 2007).

16S rRNA Gene Sequencing:

Individual samples from CIMP and Non-CIMP groups are subject to 16S rRNA gene sequencing as described in Example 1. A minimum of 10 million total 16S sequences and approximately 20,000-50,000 16S sequences per tissue sample for all 200 specimens are analyzed in this study. Based on these data, BPB deficiency in CIMP compared to Non-CIMP patient tissues can be evaluated. It is expected that additional features that differentiate the microbiota between these two tumor types will be identified. Specifically, qPCR assays are used that are frequently employed to quantitate levels of Faecalibacterium prausnitzii and Eubacterium and Roseburia spp. and are based on published assays (Louis and Flint 2007; Vital, et al. 2013; Ramirez-Farias, et al. 2009). Levels of BPB will be normalized to total bacterial levels based on a pan-bacterial 16S rDNA-based qPCR assay (Nadkarni, et al. 2002) that has been constructed. Differences between CIMP and Non-CIMP tumor and normal tissue will be determined based on the Mann-Whitney U test or ANOVA, depending on the distribution of the data.

### Composition of the Bacterial Metagenome:

PICRUSt as described in Example 1 is used to predict the full bacterial gene content from CIMP and Non-CIMP patients. PICRUSt analysis may also lead to the discovery of additional bacterial genes or functions that differentiate CIMP and Non-CIMP, including pathways involved in the synthesis of the bacterial SCFA metabolites propionate and acetate.

### Results

Using 16S rRNA sequencing, ten million total 16S sequences and approximately 20,000-50,000 16S sequences per tissue sample for all 200 specimens are analyzed in this study. Based on these data, BPB deficiency in CIMP as compared to Non-CIMP patient tissues is analyzed. Additional features that differentiate the microbiota between these two tumor types are also found.

The quantity of BPB is lower in intestinal biopsies (tumor and normal tissue) from CIMP patients as compared to Non-CIMP patients based on the levels of 16S rRNA genes for the major BPB groups. Moreover, the quantity of capacity for butyrate production is lower in CIMP patients based on the levels of BcoA DNA.

Using PICRUSt to predict the full bacterial gene content from CIMP and Non-CIMP patients, additional bacterial genes or functions that differentiate CIMP and Non-CIMP, including pathways involved in the synthesis of the bacterial SCFA metabolites propionate and acetate are identified.

### Example 5-Butyrate and Other Short Chain and Medium Chain Fatty Acids (SCFAs and MCFAs) is Specifically Reduced in CIMP Patients

In Example 1, butyrate was 8-fold lower in CIMP compared to Non-CIMP tumors and 3-fold lower in CIMP normal tissue compared to Non-CIMP normal tissue. The hypothesis is that in accord with deficiencies in BPB, the short chain fatty acid (SCFA), butyrate, is specifically reduced in tumors and normal tissue biopsies from CIMP patients and correlates with BPB deficiency and tumor methylation.

Tumor and normal tissue specimens were obtained from the Weill Cornell Colorectal Cancer Biobank (a total of 200 samples (50 CIMP tumors and matched normal tissue and 50 Non-CIMP tumors and matched normal tissue)).

Concentrations of short and medium chain fatty acids (butyrate, acetate, propionate, isovalerate, valerate, hexanoate, heptanoate, and octanoate) are measured using electron ionization-GCMS as described in Example 2. Differences between CIMP and Non-CIMP tumor and normal tissue are determined based on the Mann-Whitney U test or ANOVA, depending on the distribution of the data. Spearman's rank or Pearson's product-moment correlations will be employed to investigate the association between paired tumor and normal tissue in patients.

Given that butyrate is derived exclusively from BPB in the large intestine as a result of fiber fermentation, it is hypothesized that the relative abundance and/or quantity of tissue-associated BPB will be positively correlated with tissue-associated butyrate concentrations. In order to evaluate the relationship between BPB abundance, tissue butyrate concentrations, and methylation. the correlation (Spearman's or Pearson's correlation) between the relative abundance of BPB is determined by 16S sequencing and/or quantities of BPB determined by real-time PCR, and tissue butyrate concentrations in CIMP and Non-CIMP tumors and normal tissue are analyzed. A significant positive correlation indicates that levels of butyrate in tissue are directly related to BPB levels and are not solely related to differential metabolism or transport of butyrate in CIMP and Non-CIMP patients.

Furthermore, butyrate may have a dose effect on methylation in CIMP tumors. CIMP patients are dichotomized into sub-classifications of CIMP-high and CIMP-low based on the eight CIMP-specific promoters assessed by MethylLight PCR: CACNA1G, CDKN2A (p16), CRABP1, IGF2, MLH1, NEUROG1, RUNX3, and SOCS1. Abundance of BPB and butyrate concentrations are compared between CIMP-high tumors and CIMP-low tumors in order to determine whether methylation states are associated with BPB and butyrate. Differences between CIMP-high and CIMP-low tumors are determined based on the Mann-Whitney U test or ANOVA.

### Example 6—Investigation of the Impact of Diet-Induced Disruption of BPB in HBUS Mice

The impact of diet-induced disruption of BPB in HBUS mice is investigated. Without being bound by theory, diet-induced butyrate deficiency produced by lack of BPB can trigger progression of serrated tumorigenesis by overcoming senescence and instigating aberrant CIMP-associated methylation.

### Materials and Methods

### Mice:

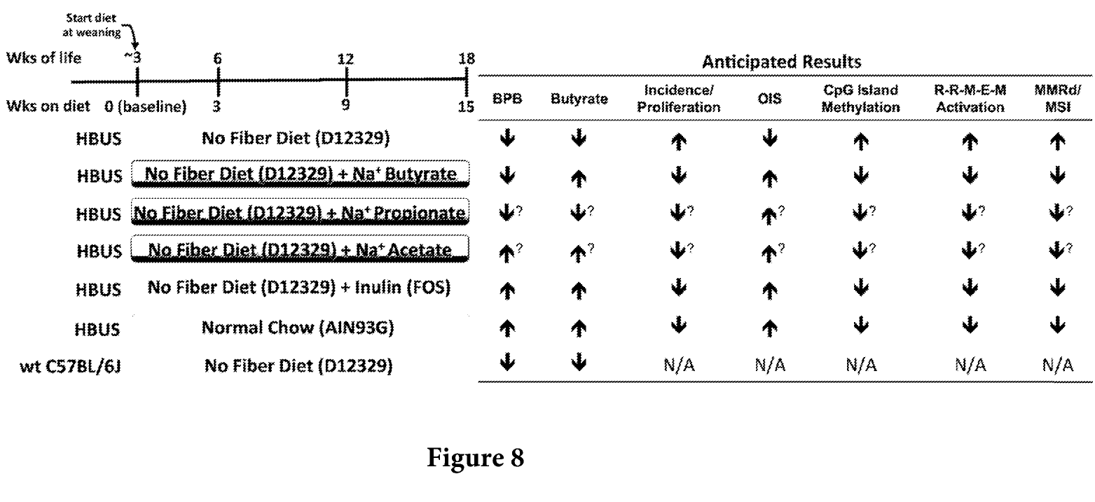

At weaning, HBUS mice are randomly divided into one of 6 dietary groups (FIG. 8). One group is placed on a fiber-deficient diet (D12329; Research Diets, Inc), representing the primary test group. A second group is placed on the same fiber-deficient diet, but this diet will be supplemented with Na+-butyrate (5% wt/wt). This test group is anticipated to have deficiencies in BPB, but a protective effect from Na+-butyrate is predicted. The third group is placed on the same fiber-deficient diet supplemented with molarity-matched Na+-propionate (4.3% wt/wt). The HDACi activity of Na+-propionate may provide a protective effect similar to that of Na+-butyrate, though likely to a lesser extent (Waldecker, et al. 2012). The fourth group is placed on the same fiber-deficient diet supplemented with molarity-matched Na+-acetate (3.7% wt/wt). As acetate is a substrate in the final reaction of the BcoA pathway of butyrate synthesis and has been shown to be essential for growth of some BPBs (Duncan, et al. 2002), Na+-acetate may stimulate the growth of BPB even in the absence of fiber. The next group is placed on the fiber-deficient diet, but this diet is supplemented with prebiotic inulin/fructooligosaccharides (inulin/FOS) (6% wt/wt) (Perrin, et al. 2001). Inulin/FOS has been shown to induce the growth of BPB in vitro and in vivo and to increase intestinal butyrate content (Perrin, et al. 2001; Scott, et al. 2014; DePreter, et al. 2013; Licht, et al. 2006; Kleessen, et al. 2001; Pool-Zobel, et al. 2005). The results describe herein in Example 2 indicated that inulin's BPB elevating effect is rapid (occurring within one week) and sustained (remaining elevated over the 9-week course). This test group is expected to have increased levels of BPB and butyrate and thus a protective effect.

Two control groups are also included. The first is a group of HBUS mice placed on a normal chow diet (the same diet used in the original description of this model (Bongers, et al. 2012)). A second control group consists of wt C57BL/6J mice (the background strain of HBUS) on the fiber-deficient diet. These mice will serve as a control for the fiber-deficient diet, as it is expected that this diet alone will not lead to the development of tumors in the absence of activating mutations in the R-R-M-E-M pathway.

The intestines, cecal contents, and feces of mice in each of the 7 groups (5 test groups and 2 control groups) are evaluated for each of the parameters described herein at each of 3 time points (6 weeks, 12 weeks, and 18 weeks) (FIG. 8). A minimum of 10-12 mice per group is evaluated at each time point. The approximate 3-week time point corresponds to weaning, which may vary between 21-28 days, determined based on the state of health of individual mice (to protect mice from adverse outcomes of early weaning). The other time points of 6 weeks, 12 weeks, and 18 weeks correspond to time points identified in the HBUS mouse study that demonstrated increasing tumor incidence with increasing age. These time points are expected to correlate with approximately 50% polyp incidence (6 weeks), approximately 5% polyp incidence (12 weeks), and greater than 86% polyp incidence (18 weeks) on a normal chow diet (Bongers, et al. 2012).

As shown in FIG. 8, various parameters are measured.

### BPB:

At each of the three time points and for each diet in the protocol, cecal contents, fecal pellets, and muco-epithelial tissue will be collected. As BPB populations may differ between mice and humans, 16S rDNA sequencing will be carried out on the MiSeq platform to evaluate cecal, fecal, and muco-epithelial-associated BPB and other taxa in mice. BPB will also be quantitated relative to total bacteria populations using cecal, fecal, and tissue-derived DNA and the 16S rDNA and BcoA-specific qPCR assays described herein in Example 1. 16S sequencing may implicate bacteria involved in propionate or acetate synthesis as being differentially abundant in HBUS mice in one of the dietary groups. qPCR assays can be designed to quantitate these bacteria in each dietary group.

### Butyrate:

Butyrate, propionate and acetate concentrations in cecal contents and feces will be determined by gas chromatography/mass spectrometry at the Irving Institute for Clinical and Translational Research Biomarkers Core Facility, Columbia University as previously described (Zumbrun, et al. 2013).

### Incidence/Proliferation:

Both small and large intestinal tumor incidence, number and size is evaluated in mice from each dietary group, at the three time points. Proliferation is assessed in isolated intestinal epithelial cells following i.p. BrdU injection, as well as in fixed intestinal tissue using Ki-67 immunostaining (Bongers, et al. 2012).

### OIS:

Butyrate may play a role in OIS maintenance in serrated tumorigenesis. Na+-butyrate has been shown to induce a senescence-like state in human cells, an effect that is dependent on p16^{Ink4a} (Munro, et al. 2004). p16^{Ink4a}-induced senescence occurs in all mouse models of serrated tumorigenesis, including HBUS mice (Bennecke, et al. 2010; Bongers, et al. 2012; Carragher, et al. 2010; Rad, et al. 2013). Butyrate produced by bacteria in the large intestine may play a fundamental role in maintaining OIS and preventing tumor progression as a result of butyrate's HDACi activity. The loss of butyrate in the colon can lead to downregulation of p16^{Ink4a} by promoting methylation of the CDKN2A promoter and could overcome OIS in HBUS mice. p16^{Ink4a} mRNA levels are quantitated by qPCR from isolated colonic enterocytes and directly from intestinal tissue from HBUS mice on the dietary regimens described above. Protein levels are investigated by western blots, and in vivo localization in tissues will be assessed by immunohistochemical/immunofluorescence methods as described (Bennecke, et al. 2010; Carragher, et al. 2010; Williams and Lipkin 2006). Other OIS markers are also evaluated, including SA-β-galactosidase staining in tissue sections and qPCR for Dec1 and p19Arf mRNA levels (Bennecke, et al. 2010).

### CpG Methylation:

CIMP may develop in serrated tumors as a result of aberrant methylation mediated by colonic butyrate deficiency. Both global and specific CpG island methylation are evaluated in DNA obtained from cecal tumors and normal tissue from HBUS mice on the diets described above. Methylated DNA immunoprecipitation (MeDip) CpG island microarray analysis (MeDip-Chip; Agilent Mouse CpG Island Microarray) will be used to evaluate global methylation profiles of CpG islands in tumor tissues from the mice MeDip, sample labeling, hybridization, and analysis will be carried out according to the manufacturer's protocols and as previously described in Bennecke, et al. 2010 and Weng, et al. 2009. Dual-labeled arrays are scanned using an Agilent compatible NimbleGen MS 200 scanner. Validation of CpG islands found to be differentially methylated in HBUS mice on fiber-deficient diets are carried out using bisulfite sequencing, COBRA, or methylation-specific PCR. Butyrate deficiency may lead to specific methylation of genes that are highly associated with CIMP in humans. In particular, butyrate deficiency may lead to methylation of the CDKN2A promoter, causing decreased expression of p16^{Ink4a} and bypass of senescence. MLH1 methylation may be increased in HBUS mice on a fiber-deficient diet, leading to mismatch repair deficiency. The CpG islands for these genes are investigated using bisulfite sequencing (Rad, et al. 2013). Previous studies suggest that HDACi may influence DNMT expression, which could play a role in CIMP development (Sarkar, et al. 2011; Xiong, et al. 2005). To investigate this, mRNA expression is evaluated by qPCR and protein levels by western blotting to determine whether levels of DNMT1, DNMT3a, or DNMT3b are differentially expressed as a result of butyrate deficiency in tumors of HBUS mice.

### R-R-M-E-M Activation:

Butyrate may influence R-R-M-E-M activation at two points in the pathway. To evaluate the impact of butyrate deficiency, EGFR protein and mRNA levels is compared, as well as EGFR phosphorylation, between dietary groups using a combination of immunofluorescence in tissue sections with Image J analysis, western blotting of isolated tumor proteins, and qPCR analysis as previously described (Bongers, et al. 2012). Similarly, ERK phosphorylation will be investigated between the dietary groups using phospho-ERK antibodies in immunofluorescence and western blotting experiments (Bennecke, et al. 2010; Bongers, et al. 2012; Carragher, et al. 2010). The Center for Infection and Immunity also uses a NanoPro 1000 (proteinsimple) to facilitate detection of phosphorylation states.

### Results

The expected results for each parameter and each group of mice are set forth in FIG. 8.

### REFERENCES

Arrington, et al., Prognostic and Predictive Roles of KRAS Mutation in Colorectal Cancer. Int. J. Mol. Sci., 2012. 13(10):12153-68.
Asanuma, et al., Characterization and transcription of the genes involved in butyrate production in Butyrivibrio fibrisolvens type I and II strains. Curr. Microbiol., 2005. 51(2):91-4.
Bachman, et al., Dnmt3a and Dnmt3b are transcriptional repressors that exhibit unique localization properties to heterochromatin. J. Biol. Chem., 2001. 276(34):32282-7.
Barcenilla, et al., Phylogenetic relationships of butyrate-producing bacteria from the human gut. Appl. Environ. Microbiol., 2000. 66(4):1654-61.
Beach, et al., BRAF mutations in aberrant crypt foci and hyperplastic polyposis. Am. J. Pathol., 2005. 166(4):1069-75.
Bennecke, et al., Ink4a/Arf and oncogene-induced senescence prevent tumor progression during alternative colorectal tumorigenesis. Cancer Cell, 2010. 18(2): p. 135-46.
Bettington, et al., The serrated pathway to colorectal carcinoma: current concepts and challenges. Histopathology, 2013. 62(3):367-86.
Biagi, et al., Through ageing, and beyond: gut microbiota and inflammatory status in seniors and centenarians. PLoS One, 2010. 5(5):e10667.
Bierne, et al., Epigenetics and bacterial infections. Cold Spring Harbor Perspect. Med. 2012. 2(12):a010272.
Bongers, et al., A role for the epidermal growth factor receptor signaling in development of intestinal serrated polyps in mice and humans. Gastroenterology, 2012. 143(3):730-40.
Bongers, et al., Interplay of host microbiota, genetic perturbations, and inflammation promotes local development of intestinal neoplasms in mice. J. Exp. Med., 2014. 211(3): p. 457-72.
Cai, et al., The NuRD complex cooperates with DNMTs to maintain silencing of key colorectal tumor suppressor genes. Oncogene, 2014. 33:2157-68.
Canani, et al., Potential beneficial effects of butyrate in intestinal and extraintestinal diseases. World J. Gastroenterol., 2011. 17(12):1519-28.
Caporaso, et al., Ultra-high-throughput microbial community analysis on the Illumina HiSeq and MiSeq platforms. ISME J., 2012. 6(8):1621-4
Carragher, et al., V600EBraf induces gastrointestinal crypt senescence and promotes tumour progression through enhanced CpG methylation of p16INK4a. EMBO Mol. Med., 2010. 2(11):458-71.
Castellarin, et al., Fusobacterium nucleatum infection is prevalent in human colorectal carcinoma. Genome Res., 2012. 22(2):299-306.
Chan, et al., BRAF and KRAS mutations in colorectal hyperplastic polyps and serrated adenomas. Cancer Res., 2003. 63(16):4878-81.
Chou, et al., HDAC inhibition decreases the expression of EGFR in colorectal cancer cells. PLoS One, 2011. 6(3):e18087.
Claesson, et al., Composition, variability, and temporal stability of the intestinal microbiota of the elderly. Proc. Natl. Acad. Sci. USA, 2011. 108 Suppl 1:4586-91.
Claesson, et al., Gut microbiota composition correlates with diet and health in the elderly. Nature, 2012. 488(7410):178-84.
Cunningham, et al., Cetuximab monotherapy and cetuximab plus irinotecan in irinotecan-refractory metastatic colorectal cancer. N. Engl. J. Med., 2004. 351(4):337-45.
Daly and Shirazi-Beechey, Microarray analysis of butyrate regulated genes in colonic epithelial cells. DNA Cell. Biol., 2006. 25(1):49-62.
DeFilippo, et al., Impact of diet in shaping gut microbiota revealed by a comparative study in children from Europe and rural Africa. Proc. Natl. Acad. Sci. USA, 2010. 107(33):14691-6.
DePreter, et al., Metabolic profiling of the impact of oligofructose-enriched inulin in Crohn's disease patients: a double-blinded randomized controlled trial. Clin. Transl. Gastroenterol., 2013. 4:e30.
Ding, et al., Alterations of MAPK activities associated with intestinal cell differentiation. Biochem. Biophys. Res. Commun., 2001. 284(2):282-8.
Duncan, et al., Acetate utilization and butyryl coenzyme A (CoA):acetate-CoA transferase in butyrate-producing bacteria from the human large intestine. Appl. Environ. Microbiol., 2002. 68(10):5186-90.
Duncan, et al., Reduced dietary intake of carbohydrates by obese subjects results in decreased concentrations of butyrate and butyrate-producing bacteria in feces. Appl. Environ. Microbiol., 2007. 73(4):1073-8.
Edgar, Search and clustering orders of magnitude faster than BLAST. Bioinformatics, 2010. 26(19):2460-1.
Hawkins, et al., CpG island methylation in sporadic colorectal cancers and its relationship to microsatellite instability. Gastroenterology, 2002. 122(5):1376-87.
Hippe, et al., Quantification of butyryl CoA:acetate CoA-transferase genes reveals different butyrate production capacity in individuals according to diet and age. FEMS Microbiol. Letters, 2011. 316(2):130-5.
Hope, et al., Sporadic colorectal cancer—role of the commensal microbiota. FEMS Microbiol. Letters, 2005. 244(1):1-7.
Jass Classification of colorectal cancer based on correlation of clinical, morphological and molecular features. Histopathology, 2007. 50(1):113-30.
Kleessen, et al., Oligofructose and long-chain inulin: influence on the gut microbial ecology of rats associated with a human faecal flora. Br. J. Nutr., 2001. 86(2):291-300.
Konstantinidis and Tiedje, Genomic insights that advance the species definition for prokaryotes. Proc. Natl. Acad. Sci. USA, 2005. 102(7):2567-72.
Kostic, et al., Genomic analysis identifies association of Fusobacterium with colorectal carcinoma. Genome Res., 2012. 22(2):292-8.
Kriegl, et al., Up and downregulation of p16(Ink4a) expression in BRAF-mutated polyps/adenomas indicates a senescence barrier in the serrated route to colon cancer. Mod. Pathol., 2011. 24(7):1015-22.
Kuczynski, et al., Using QIIME to analyze 16S rRNA gene sequences from microbial communities. Curr. Protoc. Microbiol., 2012. Chapter 1: Unit 1E 5.
Langille, et al., Predictive functional profiling of microbial communities using 16S rRNA marker gene sequences. Nature Biotechnology, 2013. 31(9):814-21.
Lee, et al., Clinicopathological features of CpG island methylator phenotype-positive colorectal cancer and its adverse prognosis in relation to KRAS/BRAF mutation. Pathol. Int., 2008. 58(2):104-13.
Leonel and Alvarez-Leite, Butyrate: implications for intestinal function. Curr. Opin. Clin. Nutr. Metab. Care, 2012. 15(5):474-9.
Licht, et al., Dietary carbohydrate source influences molecular fingerprints of the rat faecal microbiota. BMC Microbiol., 2006. 6: 98.
Louis and Flint, Development of a semiquantitative degenerate real-time pcr-based assay for estimation of numbers of butyryl-coenzyme A (CoA) CoA transferase genes in complex bacterial samples. Appl. Environ. Microbiol., 2007. 73(6):2009-12.
Louis and Flint, Diversity, metabolism and microbial ecology of butyrate-producing bacteria from the human large intestine. FEMS Microbiol. Letters, 2009. 294(1):1-8.
Louis, et al., Restricted distribution of the butyrate kinase pathway among butyrate-producing bacteria from the human colon. J. Bacteriol., 2004. 186(7):2099-106.
Louis, et al., Organization of butyrate synthetic genes in human colonic bacteria: phylogenetic conservation and horizontal gene transfer. FEMS Microbiol. Letters, 2007. 269(2):240-7.
Lozupone and Knight, UniFrac: a new phylogenetic method for comparing microbial communities. Appl. Environ. Microbiol., 2005. 71(12):8228-35.
Lozupone, et al., Meta-analyses of studies of the human microbiota. Genome Res., 2013. 23(10): 1704-14.
McCoy, et al., Fusobacterium is associated with colorectal adenomas. PLoS One, 2013. 8(1): e53653.
Minoo, et al., Extensive DNA methylation in normal colorectal mucosa in hyperplastic polyposis. Gut, 2006. 55(10):1467-74.
Munro, et al., Histone deacetylase inhibitors induce a senescence-like state in human cells by a p16-dependent mechanism that is independent of a mitotic clock. Exp. Cell. Res., 2004. 295(2):525-38.
Nadkarni, et al., Determination of bacterial load by real-time PCR using a broad-range (universal) probe and primers set. Microbiology, 2002. 148(Pt 1):257-66.
Nakajima, et al., The presence of a methylation fingerprint of Helicobacter pylori infection in human gastric mucosae. Int. J. Cancer, 2009. 124(4):905-10.
Neyrinck, et al., Dietary modulation of clostridial cluster XIVa gut bacteria (Roseburia spp.) by chitin-glucan fiber improves host metabolic alterations induced by high-fat diet in mice. J Nutr. Biochem., 2012. 23(1):51-9.
Nishihara, et al., Long-term colorectal-cancer incidence and mortality after lower endoscopy. N. Eng.l J. Med., 2013. 369(12):1095-105.
O'Brien, et al., Comparison of microsatellite instability, CpG island methylation phenotype, BRAF and KRAS status in serrated polyps and traditional adenomas indicates separate pathways to distinct colorectal carcinoma end points. Am. J. Surg. Pathol., 2006. 30(12):1491-501.
Ogino, et al., Precision and performance characteristics of bisulfite conversion and real-time PCR (MethyLight) for quantitative DNA methylation analysis. J. Mol. Diagn., 2006. 8(2):209-17.
Ogino, et al., CpG island methylator phenotype-low (CIMP-low) in colorectal cancer: possible associations with male sex and KRAS mutations. J. Mol. Diagn., 2006. 8(5):582-8.
Ogino, et al., Evaluation of markers for CpG island methylator phenotype (CIMP) in colorectal cancer by a large population-based sample. J. Mol. Diagn., 2007. 9(3):305-14.
Perrin, et al., Only fibres promoting a stable butyrate producing colonic ecosystem decrease the rate of aberrant crypt foci in rats. Gut, 2001. 48(1):53-61.
Pool-Zobel, Inulin-type fructans and reduction in colon cancer risk: review of experimental and human data. Br. J. Nutr., 2005. 93 Suppl 1:S73-90.
Qin, et al., A human gut microbial gene catalogue established by metagenomic sequencing. Nature, 2010. 464(7285):59-65.
Rad, et al., A genetic progression model of Braf(V600E)-induced intestinal tumorigenesis reveals targets for therapeutic intervention. Cancer Cell, 2013. 24(1):15-29.
Ramirez-Farias, et al., Effect of inulin on the human gut microbiota: stimulation of Bifidobacterium adolescentis and Faecalibacterium prausnitzii. Br. J. Nutr., 2009. 101(4):541-50.
Rosenberg, et al., Mutations in BRAF and KRAS differentially distinguish serrated versus non-serrated hyperplastic aberrant crypt foci in humans. Cancer Res., 2007. 67(8):3551-4.
Rountree, et al., DNA methylation, chromatin inheritance, and cancer. Oncogene, 2001. 20(24):156-65.
Sarkar, et al., Histone deacetylase inhibitors reverse CpG methylation by regulating DNMT1 through ERK signaling. Anticancer Res., 2011. 31(9):2723-32.
Sawyer, et al., Molecular characteristics of serrated adenomas of the colorectum. Gut, 2002. 51(2):200-6.
Scott, et al., Prebiotic stimulation of human colonic butyrate-producing bacteria and bifidobacteria, in vitro. FEMS Microbiol. Ecol., 2014. 87(1):30-40.
Segata and Huttenhower, Toward an efficient method of identifying core genes for evolutionary and functional microbial phylogenies. PLoS One, 2011. 6(9):e24704.
Segata, et al., Metagenomic biomarker discovery and explanation. Genome Biol., 2011. 12(6):R60.
Sepulveda, et al., CpG methylation and reduced expression of 06-methylguanine DNA methyltransferase is associated with Helicobacter pylori infection. Gastroenterology, 2010. 138(5):1836-44.
Siegel, et al. Cancer statistics, 2013. CA. Cancer. J. Clin., 2013. 63(1):11-30.
Simons, et al., A novel classification of colorectal tumors based on microsatellite instability, the CpG island methylator phenotype and chromosomal instability: implications for prognosis. Ann. Oncol., 2013. 24(8):2048-56.
Slattery, et al., Diet and lifestyle factor associations with CpG island methylator phenotype and BRAF mutations in colon cancer. Int. J. Cancer, 2007. 120(3):656-63.
Snel et al., Genome phylogeny based on gene content. Nature Genetics, 1999. 21(1):108-10.
Snover Update on the serrated pathway to colorectal carcinoma. Human Pathol., 2011. 42(1):1-10.
Stegeman, et al., Colorectal cancer risk factors in the detection of advanced adenoma and colorectal cancer. Cancer Epidemiol., 2013. 37(3):278-83.
Takahashi, et al., Epigenetic control of the host gene by commensal bacteria in large intestinal epithelial cells. J. Biol. Chem., 2011. 286(41).
Topping and Clifton, Short-chain fatty acids and human colonic function: roles of resistant starch and nonstarch polysaccharides. Physiol. Rev., 2001. 81(3):1031-64.
Toyota, et al., CpG island methylator phenotype in colorectal cancer. Proc Natl Acad Sci USA, 1999. 96(15):8681-6.
van Rijnsoever, et al., Characterisation of colorectal cancers showing hypermethylation at multiple CpG islands. Gut, 2002. 51(6):797-802.
Vaughn, et al., Quantitative evaluation of CpG island methylation in hyperplastic polyps. Mod. Pathol., 2010. 23(1):151-6.
Velho, et al., BRAF, KRAS and PIK3CA mutations in colorectal serrated polyps and cancer: primary or secondary genetic events in colorectal carcinogenesis? BMC Cancer, 2008. 8:255.
Vital, et al., A gene-targeted approach to investigate the intestinal butyrate-producing bacterial community. Microbiome, 2013. 1(1):8.
Waldecker, et al., Inhibition of histone-deacetylase activity by short-chain fatty acids and some polyphenol metabolites formed in the colon. J. Nutr. Biochem., 2008. 19(9):587-93.
Walker, et al., Dominant and diet-responsive groups of bacteria within the human colonic microbiota. ISME J., 2011. 5(2):220-30.
Wang, et al., Suppression of neurotensin receptor type 1 expression and function by histone deacetylase inhibitors in human colorectal cancers. Mol. Cancer Ther., 2010. 9(8):2389-98.
Weng et al., Methylated DNA immunoprecipitation and microarray-based analysis: detection of DNA methylation in breast cancer cell lines. Methods Mol. Biol., 2009. 590:165-76.
Weisenberger, et al., CpG island methylator phenotype underlies sporadic microsatellite instability and is tightly associated with BRAF mutation in colorectal cancer. Nature Genetics, 2006. 38(7):787-93.
Widschwendter, et al., Association of breast cancer DNA methylation profiles with hormone receptor status and response to tamoxifen. Cancer Res., 2004. 64(11):3807-13.
Williams and Lipkin, Endoplasmic reticulum stress and neurodegeneration in rats neonatally infected with borna disease virus. J. Virol., 2006. 80(17):8613-26.
Williams, et al., Impaired carbohydrate digestion and transport and mucosal dysbiosis in the intestines of children with autism and gastrointestinal disturbances. PLoS One, 2011. 6(9):e24585.
Yang, et al., BRAF and KRAS Mutations in hyperplastic polyps and serrated adenomas of the colorectum: relationship to histology and CpG island methylation status. Am. J. Surg. Pathol., 2004. 28(11):1452-9.
Yoshida, et al., Altered mucosal DNA methylation in parallel with highly active Helicobacter pylori-related gastritis. Gastric Cancer, 2013. 16:488-97.
Zaneveld, et al., Ribosomal RNA diversity predicts genome diversity in gut bacteria and their relatives. Nucleic Acids Res., 2010. 38(12):3869-79.
Zhang, C., et al., Identification of low abundance microbiome in clinical samples using whole genome sequencing. Genome Biol., 2015. 16:265.
Zumbrun, et al., Dietary choice affects Shiga toxin-producing Escherichia coli (STEC) O157:H7 colonization and disease. Proc. Natl. Acad. Sci. USA, 2013. 110(23):E2126-33.

1. A method of treating and/or preventing colorectal cancer, non-dysplastic serrated polyps, or serrated crypt foci in a subject in need thereof, comprising administering to the subject, a therapeutically effective amount of butyrate producing bacteria, dietary fiber, or a combination thereof.

2. The method of claim 1, wherein the dietary fiber is inulin fiber.

3. The method of claim 1, wherein the colorectal cancer is chosen from the group consisting of CpG island methylator phenotype (CIMP) and serrated neoplasia.

4. (canceled)

5. The method of claim 1, wherein the butyrate producing bacteria or dietary fiber is administered orally.

6. The method of claim 1, wherein the butyrate producing bacteria is administered rectally.

7. The method of claim 1, wherein the butyrate producing bacteria administered to the subject belong to clostridial cluster XIVa, clostridial cluster IV, or a combination thereof.

8. The method of claim 1, wherein the butyrate producing bacteria administered to the subject belong to the phylum Firmicutes, the phylum Bacteroidetes, or a combination thereof.

9. The method of claim 1, wherein the butyrate producing bacteria administered to the subject belong to the genus Roseburia, the genus Eubacterium, the genus Faecalibacterium, the genus Clostridium, the genus Prevotella, or a combination thereof.

10.-17. (canceled)

18. The method of claim 1, wherein the level of butyrate producing bacteria is measured in a sample from the subject before administration and the butyrate producing bacteria, dietary fiber, or a combination thereof, is administered to the subject if the level of butyrate producing bacteria in the sample from the subject is reduced compared to the level of butyrate producing bacteria in a control sample.

19. (canceled)

20. The method of claim 1, wherein the alpha diversity of bacteria is measured in a sample from the subject before administration and the butyrate producing bacteria, dietary fiber, or a combination thereof, is administered to the subject if the alpha diversity of the sample from the subject is reduced compared to the alpha diversity of a control sample.

21. (canceled)

22. The method of claim 1, wherein the operational taxonomic units (OTUs) is measured in a sample from the subject before administration and the butyrate producing bacteria, dietary fiber, or a combination thereof, is administered to the subject if the OTUs of the sample from the subject are reduced compared to the OTUs of a control sample.

23. (canceled)

24. The method of claim 18, wherein the level of butyrate producing bacteria is the level of bacteria belonging to clostridial cluster XIVa or clostridial cluster IV.

25. The method of claim 18, wherein the level of butyrate producing bacteria is the level of bacteria belonging to the phylum Firmicutes, the phylum Bacteroidetes, or a combination thereof.

26. The method of claim 18, wherein the level of butyrate producing bacteria is the level of bacteria belonging to the genus Roseburia, the genus Eubacterium, the genus Faecalibacterium, the genus Clostridium, the genus Prevotella, or a combination thereof.

27. (canceled)

28. The method of claim 18, wherein the level of butyrate producing bacteria is the level of mucin-degrading bacteria.

29. The method of claim 18, wherein the butyrate producing bacteria, dietary fiber, or a combination thereof, is administered to the subject if the level of bacteria belonging to the order Lactobacillales in the sample from the subject is increased compared to the level of bacteria belonging to the order Lactobacillales in a control sample.

30. The method of claim 1, wherein the level of bacterial butyryl coenzyme A genes (CoA), bacterial acetate CoA transferase genes (BcoA), or a combination thereof, is measured in a sample from the subject before administration and wherein the butyrate producing bacteria, dietary fiber, or a combination thereof, is administered to the subject if the level of BcoA of the sample from the subject is reduced compared to the level of BcoA of a control sample or the level of CoA of the sample from the subject is reduced compared to the level of CoA of a control sample.

31. (canceled)

32. The method of claim 30, wherein the BcoA is the BcoA gene chosen from the group consisting of Faecalibacterium prausnitzii, the genus Roseburia, the genus Eubacterium, and Clostridium sp. SS2/1.

33.-36. (canceled)

37. The method of claim 18, wherein the level of butyrate producing bacteria is measured by bacterial 16S rRNA gene sequencing, bacterial 16S rRNA qPCR, RT-PCR, or qRT-PCR.

38.-40. (canceled)

41. The method of claim 30, wherein the level of CoA or BcoA is measured by qPCR, RT-PCR, or qRT-PCR.

42. The method of claim 18, wherein the sample is feces or tissue biopsy.

43.-45. (canceled)

46. The method of claim 1, wherein the cecal levels of short chain fatty acids is measured in a sample from the subject before administration and wherein the butyrate producing bacteria, dietary fiber, or a combination thereof, is administered to the subject if the level cecal levels of short chain fatty acids in the sample from the subject is reduced compared to the level cecal levels of short chain fatty acids in a control sample.

47. (canceled)

48. The method of claim 18, wherein the control sample is from a subject not suffering from colorectal cancer, non-dysplastic serrated polyps, or serrated crypt foci.

49. (canceled)

50. A method of treating a subject with a deficiency in butyrate producing bacteria comprising administering butyrate producing bacteria, dietary fiber, or a combination thereof to a subject.

51. The method of claim 50, wherein the dietary fiber is inulin fiber.

52.-91. (canceled)
